(19) United States
(12) Patent Application Publication
(10) Pub. No.: US 2026/0201038 A1
(43) Pub. Date: Jul. 16, 2026

(54) ANTIBODY SPECIFICALLY BINDING TO B7-H3

(71) Applicant: CELLABMED INC., Seoul (KR)

(72) Inventors: Anna JU, Seoul (KR); Min-Gu KIM, Seoul (KR); Boram LEE, Seongnaim-si (KR); Sangeun LEE, Seoul (KR); Yeongha JEON, Incheon (KR); Song-Jae LEE, Seoul (KR); Seong-Won SONG, Gunpo-si (KR); Ji Chul LEE, Gwangmyeong-si (KR); Sung-Won MIN, Seoul (KR); Won Hye KA, Gwangju-si (KR); Hyeong Sun KWON, Seoul (KR); Kyung Hee RHEE, Suwon-si (KR); Gong-Deuk BAE, Seoul (KR); Nam Hyuk KIM, Seoul (KR); Jineui LEE, Hwaseong-si (KR)

(73) Assignee: CELLABMED INC., Seoul (KR)

(21) Appl. No.: 19/129,935

(22) PCT Filed: Nov. 15, 2023
(86) PCT No.: PCT/KR2023/018360

§ 371 Date: May 14, 2025

(30) Foreign Application Priority Data
Nov. 15, 2022 (KR) 10-2022-0152913
Nov. 9, 2023 (KR) 10-2023-0154552

(51) Int. Cl. C07K 16/28; A61P 35/00

(52) U.S. Cl. CPC C07K 16/2827; A61P 35/00

(57) ABSTRACT

The present disclosure relates to an anti-B7-H3 antibody or an antigen-binding fragment thereof, which binds to a specific epitope located within the amino acid sequence of a domain of B7-H3. The antibody (i) specifically binds to various types of cancer cell lines expressing B7-H3, demonstrating T cell-mediated cancer cell-killing effect, and (ii) binds to epitopes in both humans and mice, thus allowing for effective evaluation of anticancer effect in preclinical trials when developed into a therapeutic antibody.

### TECHNICAL FIELD

The present disclosure relates to an antibody specifically binding to B7-H3 or an antigen-binding fragment thereof.

### BACKGROUND ART

The global solid cancer agent market is forecast to grow by 12.5% annually on the average from about 101.0 billion dollars (about 126 trillion won) in 2021 to about 252.0 billion dollars (about 310 trillion won) in 2028. In 2028, the market size is projected to be about 52.0 billion dollars (about 65 trillion won) for non-small-cell lung cancer (NSCLC), about 46.0 billion dollars (about 57 trillion won) for breast cancer, about 21.0 billion dollars (about 26 trillion won) for prostate cancer, about 16.0 billion dollars (about 20 trillion won) for melanoma, and about 13.0 billion dollars (about 16 trillion won) for renal cell carcinoma (RCC). These five major cancers are expected to account for about 60% (Evaluate Pharma Market Explorer, 2022).

According to the data acquired through the National Cancer Registration Statistics Program in 2019, the number of cancer patients had increased annually since 2015, and the number of new cancer patients in 2019 was 254,718, which was 8,844 more (3.6% increase) than in 2018 (Ministry of Health and Welfare, 2019 National Cancer Registration Statistics Program).

The National Cancer Screening Program classifies breast cancer, stomach cancer, colon cancer, liver cancer, lung cancer and cervical cancer as six major cancers. Among the six major cancers, the incidence of lung cancer, colon cancer, liver cancer, stomach cancer, and cervical cancer has decreased or has not increased significantly in the recent 10 years. In contrast, the incidence of breast cancer has grown consistently in the last 20 years.

Breast cancer is classified by the presence of estrogen receptor, progesterone receptor and epidermal growth factor (HER2). Breast cancer is diagnosed as triple-negative breast cancer if it is tested ‘negative (not detected)’ for all the three proteins.

Whereas the five-year survival rate breast cancer is 90% or higher, it is about 70% for triple-negative breast cancer. The five-year survival rate of triple-negative breast cancer patients at stages 3 and 4 is only about 30%. This is lower than that of the patients with breast cancer other than triple-negative breast cancer at stages 3 and 4, which is about 50%. The progress of triple-negative breast cancer is fast and aggressive, with about 30% of early detected patients experiencing the risk of death within 5 years of surgical operation.

Triple-negative breast cancer has many limitations in treatment since it shows many mutations of cancer cells and rapid metastasis to other organs compared to other breast cancer subtypes. In addition, triple-negative breast cancer is associated with poor prognosis, with approximately 50% of patients experiencing disease recurrence despite early treatment. Currently, therapeutic agents for breast cancer target estrogen, progesterone and epidermal growth factor receptors. Accordingly, it is difficult to expect therapeutic effect from the drugs for triple-negative breast cancer patients who are negative for these receptors.

In addition to adult cancer, childhood cancer is also occurring a lot. The incidence of childhood cancer is similar globally. In Korea, approximately 1,000 to 1,200 new cases of childhood cancer are diagnosed each year, corresponding to about one in every 100,000 children, and accounting for roughly 1% of total cancer patients. Childhood cancer is the number one cause of death in children aged between 1 and 9 years, surpassing even traffic accidents.

Typical childhood cancers include leukemia, lymphoma and brain tumor. Lymphoblastic leukemia, in which lymphoid leukocytes do not mature properly but turn into cancer cells, shows excellent therapeutic outcomes, with a cure rate of approximately 80-90%. Acute myeloid leukemia also shows a cure rate of 70%. However, for pediatric brain tumor, a new therapy needs to be developed because of poor prognosis. According to data from the Korea Central Cancer Registry of Ministry of Health and Welfare, 247,952 cases of new cancer occurred in Korea in 2020, and the cases of brain tumor were 1,795, which corresponds to 0.7% of all cancers. Of these, 152 cases were reported as pediatric brain tumors—74 cases occurred in patients aged 10 to 19 years, and 78 cases in patients aged 9 years or younger. It occurred more frequently in males, with the male-to-female ratio of 1.8:1. According to the National Cancer Information Center, the brain tumors commonly occurring in children are medulloblastoma, astrocytoma, ependymoma, craniopharyngioma, brainstem glioma and germ cell tumor. The brain tumors can be classified based on malignancy, site of origin, and cell type.

Among them, medulloblastoma originates in the central part of the cerebellum and frequently metastasizes through the cerebrospinal fluid. It shows the symptoms of increased brain pressure, such as headache, motor disturbance, cranial nerve palsy, disorder of consciousness, etc., and is the most common malignant brain tumor in children. Among the brain tumors, brainstem glioma is a glioma occurring in the midbrain, pons and medulla oblongata of the brain. It causes multiple cranial nerve disorders such as dysarthria, dysphagia, squint, facial nerve paralysis, etc. or hemiparesis. It is pediatric brain tumor with the worst prognosis, with a 2-year survival rate of below 10%. At present, brain tumor is treated basically by surgery or radiation therapy, and anticancer chemotherapy is often attempted depending on the tumor tissues.

For surgical treatment of medulloblastoma, cerebellar mutism syndrome (CMS) occurs in 25% of patients. Cerebellar mutism syndrome is a condition that may occur in patients who have received surgery to remove tumor in a specific region of the brain including the cerebellum. Symptoms appear within about 1-2 days after surgery, and include loss of speech, loss of balance, trouble walking, loss of muscle tone, etc. Many of the symptoms of cerebellar mutism syndrome go away over time. There is also difficulty in performing radiation therapy after surgery. After surgery, craniospinal radiation therapy is used to prevent the spread of medulloblastoma to the cerebrospinal fluid (CSF). Craniospinal radiation therapy improves survival without doubt, but, unfortunately, those who have received radiation therapy experience decrease in their intelligence quotients (IQ), losing 2-4 points per year.

Leptomeningeal metastatic cancer is known to account for about 5-20% of all solid cancers, mostly lung cancer, breast cancer, and melanoma. The proportion of the patients showing metastasis to the meninges is also high for lung cancer, breast cancer, and melanoma (Cancers (Basel). 2021; 13). According to the 2020 National Cancer Registration Statistics Program, 28,949 patients were diagnosed as lung cancer and 24,923 patients were diagnosed as breast cancer in 2020. It is expected that there will be at least 2,500 patients in Korea who experience metastasis to the meninges for the two cancers, and there will be more when considering the number of patients from other indications and the development of diagnostic methods and therapeutic agents. However, the development of effective therapeutic agents is not easy as compared to the increasing number of patients. Although administration of an anticancer agent such as methotrexate into the cerebral ventricle or topical radiation therapy may be helpful in improving symptoms, the prognosis is not very good, with a median overall survival of 6 months or less (Korean Journal of Medicine. 2011; Vol. 81: No. 3). Although targeted therapeutic agents that effectively cross the blood-brain barrier, such as Tagrisso, have been recently known to be effective for leptomeningeal metastatic cancer, the number of applicable patients is limited and the targeted therapeutic agents have the problem of tolerance (Neurotherapeutics. 2022; 19:1782-1798).

Meanwhile, it is known that B7-H3 is highly expressed in various types of cancers, including pediatric brain cancer, leptomeningeal metastatic cancer, and triple-negative breast cancer, and the overexpression of B7-H3 is associated with adverse clinical therapeutic outcome. According to some researches, when a human triple-negative breast cancer cell line was treated with a monoclonal antibody binding specifically to B7-H3, the growth of the triple-negative breast cancer cells was inhibited. Therefore, the inventors of the present disclosure have identified the relationship of B7-H3, which is highly expressed in cancer, and various types of cancers, and the potential of B7-H3 as a therapeutic agent for triple-negative breast cancer, etc., and have completed the present disclosure.

The matters described in this background section is only for improving the understanding of the background of the present disclosure, and it should not be accepted as acknowledging that they are well known to those having ordinary knowledge in the art.

### DISCLOSURE

### Technical Problem

The inventors of the present disclosure have intended to develop an antibody which targets B7-H3 highly expressed in solid cancers. As a result, they have completed the present disclosure by discovering an anti-B7-H3 antibody which mediates T cell-dependent cytotoxicity against cancer cells by specifically binding to various types of cancer cell lines expressing B7-H3.

Accordingly, the present disclosure is directed to providing an anti-B7-H3 antibody or an antigen-binding fragment thereof, which specifically binds to B7-H3.

The present disclosure is also directed to providing an anti-B7-H3 antibody or an antigen-binding fragment thereof, which binds to a specific epitope located within the amino acid sequence of a domain of B7-H3.

The present disclosure is also directed to providing a nucleic acid encoding the anti-B7-H3 antibody or the antigen-binding fragment thereof.

The present disclosure is also directed to providing a vector including the nucleic acid.

The present disclosure is also directed to providing a cell including the vector.

The present disclosure is also directed to providing a cell transformed with the vector.

The present disclosure is also directed to providing a composition containing an ingredient selected from the anti-B7-H3 antibody or the antigen-binding fragment thereof, a nucleic acid encoding the antibody or the antigen-binding fragment thereof, a vector including the nucleic acid, and a cell including the vector or transformed with the vector.

The present disclosure is also directed to providing a pharmaceutical composition for preventing or treating cancer, which contains an ingredient selected from the anti-B7-H3 antibody or the antigen-binding fragment thereof, a nucleic acid encoding the antibody or the antigen-binding fragment thereof, a vector including the nucleic acid, and a cell including the vector or transformed with the vector, and a pharmaceutically acceptable carrier.

The present disclosure is also directed to providing a method for treating cancer, which includes a step of administering an ingredient selected from the anti-B7-H3 antibody or the antigen-binding fragment thereof, a nucleic acid encoding the antibody or the antigen-binding fragment thereof, a vector including the nucleic acid, and a cell including the vector or transformed with the vector to a subject in need thereof.

The present disclosure is also directed to providing a composition for diagnosing cancer, which contains an ingredient selected from the anti-B7-H3 antibody or the antigen-binding fragment thereof, a nucleic acid encoding the antibody or the antigen-binding fragment thereof, a vector including the nucleic acid, and a cell including the vector or transformed with the vector.

Other objects and advantages of the present disclosure will become more apparent by the following detailed description, claims, and drawings.

### Technical Solution

According to an aspect of the present disclosure, the present disclosure provides an anti-B7-H3 antibody or an antigen-binding fragment thereof, which specifically binds to B7-H3.

B7 family proteins, which are expressed on the surface of antigen-presenting cell (APCs) including cancer cells, regulate T cell activation through interaction with proteins on the surface of T cells. B7-H3, which is a member of the B7 family proteins expressed on APCs, is known as an immune checkpoint protein that inhibits the action of T cells (Front Immunol. 2021; 12:701006). Since the B7 family proteins regulate the action of T cells through different mechanisms, the specificity of an antibody targeting B7-H3 for B7-H3 is very important.

In the present specification, the term “antibody” refers to a molecule having an antigen-binding site for specific binding to an antigen. In the present specification, the term includes a full-length antibody, any antigen-binding fragment thereof (i.e., an “antigen-binding moiety”), or a single chain thereof. In a specific exemplary embodiment, the “antibody” refers to a glycoprotein including at least two heavy chains (H) and light chains (L) connected by disulfide bonds, or an antigen-binding portion thereof. In another specific exemplary embodiment, the “antibody” refers to a single-chain antibody including a single variable domain, e.g., a V_{H}H domain. Each heavy chain consists of a heavy chain variable region (abbreviated as V_{H}) and a heavy chain constant region. In certain naturally occurring antibodies, the heavy chain constant region consists of three domains, C_{H}1, C_{H}2 and C_{H}3. In certain naturally occurring antibodies, each light chain consists of a light chain variable region (abbreviated as V_{L}) and a light chain constant region. The constant region the light chain comprises a single domain, C_{L}.

The V_{H} and V_{L} regions can be further subdivided into regions of hypervariability, termed complementarity-determining regions (CDRs), which are interspersed with regions that are more conserved, termed framework regions (FRs). Each V_{H} and V_{L} is composed of three CDRs and four FRs, arranged from the amino-terminus to the carboxyl-terminus in the order of FR1, CDR1, FR2, CDR2, FR3, CDR3 and FR4. The variable regions of the heavy chain and the light chain contain a binding domain that interacts with an antigen. The constant region of the antibody may mediate the binding of an immunoglobulin to host tissues or factors, including various cells of the immune system (e.g., effector cells) and the first complement (C1q) of the classical complement system.

The antibody can be of any type (e.g., IgG, IgE, IgM, IgD, IgA or IgY), any class (e.g., IgD, IgG2, IgG3, IgG4, IgA1 or IgA2), or any subclass (e.g., IgG1, IgG2, IgG3 and IgG4 in human; and IgG1, IgG2a, IgG2b and IgG3 in mouse) of an immunoglobulin molecule. The immunoglobulin, e.g., IgG1, exists as several allotypes which differ from by only a few amino acids. The antibody disclosed in the present specification may be derived from any of commonly known isotypes, classes, subclasses or allotypes. In a specific exemplary embodiment, the antibody disclosed in the present specification belongs to the IgG1, IgG2, IgG3 or IgG4 subclass, or any hybrid thereof. In a specific exemplary embodiment, the antibody belongs to the human IgG1, human IgG2 or human IgG4 subclass.

The “antibody” includes, by way of examples, naturally occurring and non-naturally occurring antibodies; monoclonal and polyclonal antibodies; chimeric and humanized antibodies; human and non-human antibodies, fully synthetic antibodies; single-chain antibodies; monospecific antibodies; multispecific antibodies (including bispecific antibodies); tetrameric antibodies including two heavy chain and two light chain molecules; antibody light chain monomers; antibody heavy chain monomers; antibody light chain dimers; antibody heavy chain dimers; antibody light chain-antibody heavy chain pairs; intrabodies; heteroconjugate antibodies; monovalent antibodies; camelid antibodies; affibodies; anti-idiotypic (anti-Id) antibodies (including, e.g., anti-anti-Id antibodies); and single-domain antibodies (sdAbs), which are composed of a single variable domain (e.g., VH or VL) fully capable of antigen binding to antigens (Harmen M. M. and Haard H. J. Appl Microbiol Biotechnol. 77(1): 13-22 (2007)).

The term “antigen-binding moiety” i.e., “antigen-binding fragment”, of an antibody, as used herein, refers to one or more fragments of an antibody that retain the ability to specifically bind to an antigen (e.g., domain of human Ig-like-V-type 1 or Ig-like-V-type 2). Such “fragments” are, for example, between about 8 and about 1,500 amino acids in length, suitably between about 8 and about 745 amino acids in length, more suitably about 8 to about 300, for example about 8 to about 200 amino acids, or about 10 to about 50 or 100 amino acids in length. It has been shown that the antigen-binding function of an antibody can be exerted by the fragments of a full-length antibody. Examples of binding fragments encompassed within the term “antigen-binding moiety” or “antigen-binding fragment” of an antibody, e.g., an anti-B7-H3 antibody described herein, include (i) an Fab fragment, a monovalent fragment consisting of V_{L}, V_{H}, C_{L} and C_{H}1 domains; (ii) an F(ab′)₂ fragment, a bivalent fragment consisting of two Fab fragments linked by a disulfide bridge at the hinge region; (iii) an Fd fragment consisting of V_{H} and C_{H}1 domains; (iv) an Fv fragment consisting of the V_{L} and V_{H} domains of a single arm of an antibody, and disulfide-linked Fvs (sdFv); (v) a dAb fragment consisting of a V_{H} domain (Ward el al., (1989) Nature 341:544-546); and (vi) an isolated complementarity determining region (CDR); or (vii) a combination of two or more isolated CDRs which can optionally be joined by a synthetic linker, without being limited thereto. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fv (scFv)) (see, e.g., Bird el al, (1988) Science 242:423-426; and Huston et al, (1988) Proc. Natl. Acad. Sci. USA 85:5879-5883). Such single-chain antibodies are also intended to be encompassed within the term “antigen-binding moiety” or “antigen-binding fragment” of an antibody. These antibody fragments are obtained using conventional techniques known to those with skill in the art, and the fragments are screened for utility in the same manner as are intact antibodies. The antigen-binding fragments can be produced by recombinant DNA techniques, or by enzymatic or chemical cleavage of intact immunoglobulins.

As used herein, the terms “variable region” or “variable domain” are used interchangeably in the art. The variable region typically refers to a fragment of an antibody, generally, a fragment of a light or heavy chain, typically approximately 110 to 120 amino acids in the mature heavy chain and approximately 90 to 115 amino acids in the mature light chain, which differ extensively in sequence among antibodies and are used in the binding and specificity of a particular antibody for its particular antigen. The variability in sequence is concentrated in those regions called complementarity-determining regions (CDRs) while the more highly conserved regions in the variable domain are called framework regions (FRs).

It is believed that the CDRs of the light and heavy chains are primarily responsible for the interaction and specificity of the antibody with an antigen. In a specific exemplary embodiment, the variable region is a human variable region. In a specific exemplary embodiment, the variable region includes rodent or murine CDRs and human framework regions (FRs). In a specific exemplary embodiment, the variable region is a primate (e.g., non-human primate) variable region. In a specific exemplary embodiment, the variable region includes rodent or murine CDRs and primate (e.g., non-human primate) framework regions (FRs).

As used herein, the term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to any distinct type, e.g., kappa (κ) or lambda (Λ) based on the amino acid sequence of the constant domains. Light chain amino acid sequences are well known in the art. In a specific exemplary embodiment, the light chain is a human light chain.

In the present specification, the terms “V_{L}” and “V_{L} domain” are used interchangeably to refer to the light chain variable region of an antibody.

The terms “V_{H}” and “V_{H} domain” are used interchangeably to refer to the heavy chain variable region of an antibody.

As used herein, the term “constant region” and “constant domain” are interchangeable and have the meaning common in the art. The constant domain is an antibody region, e.g., a carboxyl terminal region of a light chain and/or a heavy chain which is not directly involved in binding of the antibody to an antigen but can exhibit various effector functions, such as interaction with the Fc receptor. The constant region of an immunoglobulin molecule generally has a more conserved amino acid sequence relative to the variable domain of an immunoglobulin.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region includes the constant region of an antibody excluding the first constant region immunoglobulin domain (e.g., C_{H}1 or C_{L}). In IgG, IgA and IgD antibody isotypes, the Fc region includes two identical protein fragments, derived from the second (C_{H}2) and third (C_{H}3) constant domains of the antibody's two heavy chains. IgM and IgE Fc regions include three heavy chain constant domains (C_{H} domains 2-4) in each polypeptide chain. For IgG, the Fc region includes immunoglobulin domains Cγ2 and Cγ3 and a hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxyl-terminus of the heavy chain, wherein the numbering is according to the EU index as in Kabat. The C_{H}2 domain of a human IgG Fc region extends from about amino acid 231 to about amino acid 340, whereas the C_{H}3 domain is positioned on C-terminal side of a C_{H}2 domain in an Fc region, i.e., it extends from about amino acid 341 to about amino acid 447 of an IgG. The Fc region can be a native sequence Fc, including any allotypic variant, or a variant Fc (e.g., a non-naturally-occurring Fc). In addition, Fc can also refer to this region in isolated state or in the context of an Fc-containing protein polypeptide such as a “binding protein containing an Fc region,” also referred to as an “Fc fusion protein” (e.g., an antibody or immunoadhesion).

The terms “hinge”, “hinge domain”, “hinge region”, or “antibody hinge region” are used interchangeably and refer to the domain of a heavy chain constant region that joins the C_{H}1 domain to the C_{H}2 domain and includes the upper, middle, and lower regions of the hinge (Roux et al, J. Immunol. 1998 161:4083). The hinge provides varying levels of flexibility between the binding and effector regions of an antibody and also provides sites for intermolecular disulfide bonding between two heavy chain constant regions. The sequences of wild-type IgG1, IgG2, IgG3 and IgG4 hinges are known in the art (see, e.g., Kabat E A et al, (1991) Sequences of Proteins of Immunological Interest, Fifth Edition, U.S. Department of Health and Human Services, NIH Publication No. 91-3242; Vidarsson G. et al. Front Immunol. 5:520 (published online Oct. 20, 2014)).

As used herein, the term “isotype” refers to the antibody class (e.g., IgG1, IgG2, IgG3, IgG4, IgM, IgAQ1, IgA2, IgD and IgE antibodies) that is encoded by the heavy chain constant region genes.

An “allotype” refers to a naturally-occurring variant within a specific isotype group, which differs in a few amino acids (see, e.g., Jefferis et al, (2009) mAbs 1:1). The antibody described herein can be of any allotype. The allotypes of IgG1, IgG2, IgG3 and IgG4 are known in the art (see, e.g., Kabat E A et al, (1991) supra, Vidarsson G. et al. Front Immunol. 5:520 (published online Oct. 20, 2014); and Lefranc M P, mAbs 1:4, 1-7(2009)).

The phrases “an antibody recognizing an antigen” and “an antibody specific for an antigen” are used interchangeably herein with the term “an antibody which binds specifically to an antigen.”

In the present specification, an “isolated antibody” refers to an antibody which is substantially free of other antibodies having different antigenic specificities (e.g., an isolated antibody that specifically binds to B7-H3 is substantially free of antibodies that specifically bind antigens other than B7-H3). An isolated antibody that specifically binds to an epitope of B7-H3 can, however, have cross-reactivity to other B7-H3 proteins from different species.

“Binding affinity” generally refers to the strength of the sum total of non-covalent interactions between a single binding site of a molecule (e.g., an antibody) and its binding partner (e.g., an antigen). Unless indicated otherwise, as used herein, the “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). The affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE®, or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE®, KinExA® 3000 instrument (Sapidyne Instruments, Boise, ID), or other assays known in the art.

Antibodies typically bind specifically to their cognate antigens with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, specifically 10⁻⁸ M or less, even more specifically 10⁻⁹ M or less, and most specifically between 10⁻¹⁰ M and 10⁻¹¹ M or less, when determined by, e.g., immunoassays (e.g., ELISA) surface plasmon resonance (SPR) technology in a BIACORE™ 2000 instrument using the predetermined antigen, but does not bind with high affinity to unrelated antigens.

As used herein, the term “antigen” refers to any natural or synthetic immunogenic substance, such as a protein, peptide, or hapten. An antigen can be B7-H3 or a fragment thereof.

As used herein, an “epitope” is a term in the art and refers to a localized region of an antigen to which an antibody can specifically bind. An epitope can be, for example, contiguous amino acids of a polypeptide (linear or contiguous epitope), or an epitope can, for example, be a combination of two or more non-contiguous regions of a polypeptide or polypeptides (conformational, non-linear, discontinuous, or non-contiguous epitope). An epitope formed from contiguous amino acids is typically, but not always, retained on exposure to a denaturing solvent, whereas an epitope formed by tertiary folding is typically lost on treatment with a denaturing solvent. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides are tested for reactivity with a given antibody (e.g., anti-B7-H3 antibody). Methods for determining the spatial conformation of an epitope include techniques in the art and those described herein, for example, X-ray crystallography, 2-dimensional nuclear magnetic resonance, and HDX-MS (see, e.g., Epitope Mapping Protocols in Methods in Molecular Biology, Vol. 66, G. E. Morris, Ed. (1996)).

The term “binds to the same epitope” used for two or more antibodies means that the antibodies bind to the same segment of amino acid residues, as determined by a given method. Techniques for determining whether antibodies bind to the “same epitope on B7-H3” with the antibodies described herein include, for example, epitope mapping methods, such as, X-ray analyses of crystals of antigen-antibody complexes which provides atomic resolution of the epitope, and hydrogen/deuterium exchange mass spectrometry (HDX-MS). Other methods monitor the binding of the antibody to antigen fragments or mutated variations of the antigen, where loss of binding due to a modification of an amino acid residue within the antigen sequence is often considered an indication of an epitope component. In addition, computational combinatorial methods for epitope mapping can also be used. These methods rely on the ability of the antibody of interest to selectively bind to specific short peptides from combinatorial phage display peptide libraries. Antibodies having the same V_{H} and V_{L} or the same CDR1, 2 and 3 sequences are expected to bind to the same epitope.

Antibodies that “cross-compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In a specific exemplary embodiment, an antibody competes with, and inhibits binding of another antibody to a target by at least 50%, 60%, 70%, 80%, 90% or 100%. The level of inhibition or competition can be different depending on which antibody is the “blocking antibody” (i.e., a cold antibody that has been incubated first with the target). Competition assays can be conducted as described, for example, in Ed Harlow and David Lane, Cold Spring Harb Protoc; 2006; doi: 10.1 10I/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or adjacent epitopes (e.g., as evidenced by steric hindrance).

The term “reference antibody” refers to an antibody which is used as a reference in the assay of competing antibodies that bind to the same epitope, an overlapping epitope or adjacent epitopes. It cross-competes with the competing antibodies for binding to the epitopes.

Competitive binding assays include solid phase direct or indirect radioimmunoassay (RIA), solid phase direct or indirect enzyme immunoassay (EIA), sandwich competition assay (see Stahli et al, Methods in Enzymology 9:242 (1983)); solid phase direct biotin-avidin EIA (see Kirkland et al, J. Immunol. 137:3614 (1986)); solid phase direct labeling assay, solid phase direct labeling sandwich assay (see Harlow and Lane, Antibodies: A Laboratory Manual, Cold Spring Harbor Press (1988)); solid phase direct label RIA using 1-125 label (see Morel et al, Mol. Immunol. 25 (1): 7 (1988)); solid phase direct biotin-avidin EIA (Cheung et al, Virology 176:546 (1990)); and direct labeling RIA (Moldenhauer et al, Scand. J. Immunol 32:77 (1990)).

The term “monoclonal antibody,” as used herein, refers to an antibody or a composition of antibodies, which display single binding specificity and affinity for a particular epitope. Accordingly, the term “human monoclonal antibody” refers to an antibody or antibody composition that displays single binding specificity and which has variable and optional constant regions derived from human germline immunoglobulin sequences. In a specific exemplary embodiment, human monoclonal antibodies are produced by a hybridoma which includes a B cell obtained from a transgenic non-human animal, e.g., a transgenic mouse, having a genome including a human heavy chain transgene and a light chain transgene fused to an immortalized cell and/or by a recombinant, combinatorial human antibody library.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies include variable and constant regions that utilize particular human germline immunoglobulin sequences which are encoded by the germline genes, but include subsequent rearrangements and mutations which occur, for example, during antibody maturation. As known in the art (see, e.g., Lonberg (2005) Nature Biotech. 23(9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody for the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human antibody” (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

The term “humanized antibody” refers to an antibody in which some, most or all of the amino acids outside the CDR domains of a non-human antibody are replaced with corresponding amino acids derived from human immunoglobulins. In a specific exemplary embodiment of a humanized form of an antibody, some, most or all of the amino acids outside the CDR domains are replaced with amino acids from human immunoglobulins, whereas some, most or all amino acids within one or more CDR regions remain unchanged. Additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

The term “cross-reacts,” as used herein, refers to the ability of an antibody described herein to bind to B7-H3 of a different species. For example, an antibody described herein that binds to human B7-H3 can also bind to B7-H3 of another species (e.g., mouse B7-H3). The cross-reactivity can be measured by detecting specific reactivity with a purified antigen in binding assays (e.g., SPR or ELISA), or by detecting binding to, or functional interaction with, cells physiologically expressing B7-H3. Methods for determining cross-reactivity include standard binding assays as described herein, for example, by BIACORE® surface plasmon resonance (SPR) analysis using a BIACORE® 2000 SPR instrument (Biacore AB, Uppsala, Sweden), or flow cytometric techniques.

The term “conservative amino acid substitution” refers to substitution of an amino acid residue with an amino acid residue having a similar side chain. Families of amino acid residues having similar side chains have been defined in the art. These families include amino acids with basic side chains (e.g., lysine, arginine and histidine), acidic side chains (e.g., aspartic acid and glutamic acid), uncharged polar side chains (e.g., glycine, asparagine, glutamine, serine, threonine, tyrosine, cysteine and tryptophan), nonpolar side chains (e.g., alanine, valine, leucine, isoleucine, proline, phenylalanine and methionine), beta-branched side chains (e.g., threonine, valine and isoleucine) and aromatic side chains (e.g., tyrosine, phenylalanine, tryptophan and histidine). In a specific exemplary embodiment, a predicted nonessential amino acid residue in an anti-B7-H3 antibody is replaced with another amino acid residue from the same side chain family. Methods of identifying nucleotide and amino acid conservative substitutions which do not eliminate antigen binding are well-known in the art (see, e.g., Brummell et al, Biochem. 32:1180-1187 (1993); Kobayashi et al. Protein Eng. 12 (10): 879-884 (1999); and Burks et al. Proc. Natl. Acad. Sci. USA 94: 412-417 (1997)).

The term “substantial identity” indicates that two nucleic acids or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6 or 4 and a length weight of 1, 2, 3, 4, 5 or 6.

The nucleic acid and protein sequences described herein can further be used as a “query sequence” to perform a search in public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17): 3389-3402. When utilizing the BLAST and Gapped BLAST programs, the default parameters of the respective programs (e.g., XBLAST and NBLAST) can be used (see worldwideweb.ncbi.nlm.nih.gov).

According to a specific exemplary embodiment of the present disclosure, the anti-B7-H3 antibody or the antigen-binding fragment thereof of the present disclosure binds to an epitope located within the amino acid sequence of Ig-like-V-type, Ig-like-V-type 1 and/or Ig-like-V-type 2 domains, which are domains of B7-H3.

According to an exemplary embodiment of the present disclosure, the anti-B7-H3 antibody or the antigen-binding fragment thereof of the present disclosure binds to an epitope located within the amino acid sequence of a domain of B7-H3, which is selected from a group consisting of the sequence of Ig-like-V-type 1 of SEQ ID NO: 36, the Ig-like-V-type 2 sequence of SEQ ID NO: 38, and the Ig-like-V-type sequence of SEQ ID NO: 40.

According to a specific exemplary embodiment of the present disclosure, the anti-B7-H3 antibody or the antigen-binding fragment thereof of the present disclosure binds to one or more of amino acid residues 2 to 6 of SEQ ID NO: 36 (i.e., EVQVP), amino acid residues 6 to 10 of SEQ ID NO: 38 (i.e., EVQVP), and amino acid residues 2 to 6 of SEQ ID NO: 40 (i.e., EVQVS).

According to an exemplary embodiment of the present disclosure, since the anti-B7-H3 antibody or the antigen-binding fragment thereof exhibits T cell-mediated cancer cell-killing effect by specifically binding to both an epitope located within the amino acid sequences of human-derived Ig-like-V-type 1 and/or Ig-like-V-type 2 domains (e.g., EVQVP) and an epitope located within the amino acid sequences of a mouse-derived Ig-like-V-type domain (e.g., EVQVS) (Examples 8 and 9), it is expected to exhibit effective anticancer effect in preclinical trials when developed into a therapeutic antibody.

According to a specific exemplary embodiment of the present disclosure, the anti-B7-H3 antibody or the antigen-binding fragment thereof of the present disclosure includes light chain CDR1, CDR2 and CDR3 and heavy chain CDR1, CDR2 and CDR3, wherein the light chain CDR1, CDR2 and CDR3 have SEQ ID NOS: 3, 4 and 5, respectively, and the heavy chain CDR1, CDR2 and CDR3 have SEQ ID NOS: 6, 7 and 8, respectively.

According to a specific exemplary embodiment of the present disclosure, the anti-B7-H3 antibody or the antigen-binding fragment thereof of the present disclosure includes a light chain variable region and a heavy chain variable region.

The light chain variable region may include an amino acid sequence which is identical to the sequence represented by SEQ ID NO: 9 by at least 80%, at least 85%, at least 90%, at least 95%, at least 96%, at least 97%, at least 98%, at least 99%, or 100%.

The light chain variable region may include the light chain CDR1, CDR2 and CDR3 represented by SEQ ID NOS: 3, 4 and 5, and may include an amino acid sequence which is identical to the sequence represented by SEQ ID NO: 9 by at least 80%, at least 85%, at least 90%, at least 95%, at least 96%, at least 97%, at least 98%, at least 99%, or 100%.

The heavy chain variable region may include an amino acid sequence which is identical to the sequence represented by SEQ ID NO: 10 by at least 80%, at least 85%, at least 90%, at least 95%, at least 96%, at least 97%, at least 98%, at least 99%, or 100%.

The heavy chain variable region may include the heavy chain CDR1, CDR2 and CDR3 represented by SEQ ID NOS: 6, 7 and 8, and may include an amino acid sequence which is identical to the sequence represented by SEQ ID NO: 10 by at least 80%, at least 85%, at least 90%, at least 95%, at least 96%, at least 97%, at least 98%, at least 99%, or 100%.

According to a specific exemplary embodiment of the present disclosure, the anti-B7-H3 antibody or the antigen-binding fragment thereof of the present disclosure includes a light chain variable region represented by SEQ ID NO: 9.

According to a specific exemplary embodiment of the present disclosure, the anti-B7-H3 antibody or the antigen-binding fragment thereof of the present disclosure includes a heavy chain variable region represented by SEQ ID NO: 10.

In addition, the light chain variable region may include an amino acid sequence which is identical to the sequence represented by SEQ ID NO: 21 by at least 80%, at least 85%, at least 90%, at least 95%, at least 96%, at least 97%, at least 98%, at least 99%, or 100%.

The light chain variable region may include light chain CDR1, CDR2 and CDR3 represented by SEQ ID NOS: 3, 4 and 5, and may include an amino acid sequence which is identical to the sequence represented by SEQ ID NO: 21 by at least 80%, at least 85%, at least 90%, at least 95%, at least 96%, at least 97%, at least 98%, at least 99%, or 100%.

The heavy chain variable region may include an amino acid sequence which is identical to the sequence represented by SEQ ID NO: 22 by at least 80%, at least 85%, at least 90%, at least 95%, at least 96%, at least 97%, at least 98%, at least 99%, or 100%.

The heavy chain variable region may include heavy chain CDR1, CDR2 and CDR3 represented by SEQ ID NOS: 6, 7 and 8, and may include an amino acid sequence which is identical to the sequence represented by SEQ ID NO: 22 by at least 80%, at least 85%, at least 90%, at least 95%, at least 96%, at least 97%, at least 98%, at least 99%, or 100%.

According to a specific exemplary embodiment of the present disclosure, the anti-B7-H3 antibody or the antigen-binding fragment thereof of the present disclosure includes a light chain variable region represented by SEQ ID NO: 21.

According to a specific exemplary embodiment of the present disclosure, the anti-B7-H3 antibody or the antigen-binding fragment thereof of the present disclosure includes a heavy chain variable region represented by SEQ ID NO: 22.

According to a specific exemplary embodiment of the present disclosure, the anti-B7-H3 antibody or the antigen-binding fragment thereof of the present disclosure cross-competes with a reference antibody including a light chain variable region represented by SEQ ID NO: 9 and a heavy chain variable region represented by SEQ ID NO: 10 for binding to the epitope.

According to a specific exemplary embodiment of the present disclosure, the anti-B7-H3 antibody or the antigen-binding fragment thereof of the present disclosure cross-competes with a reference antibody including a light chain variable region represented by SEQ ID NO: 21 and a heavy chain variable region represented by SEQ ID NO: 22 for binding to the epitope.

According to another aspect of the present disclosure, the present disclosure provides a nucleic acid encoding the anti-B7-H3 antibody or the antigen-binding fragment thereof.

In the present specification, the term “nucleic acid molecule” has a meaning that generally encompasses DNA (gDNA and cDNA) and RNA molecules, and nucleotides, which are the basic units of the nucleic acid molecule, includes not only natural nucleotides but also their analogues in which the sugar or base moiety is modified (Scheit, Nucleotide Analogs, John Wiley, New York (1980); Uhlman and Peyman, Chemical Reviews, (1990) 90:543-584). The sequence of the nucleic acid molecule encoding the heavy chain and light chain variable regions of the present disclosure may be modified. The modification may include the addition, deletion, or non-conservative substitution or conservative substitution of nucleotides.

The nucleic acid molecule of the present disclosure is interpreted to include a nucleotide sequence exhibiting substantial identity to the nucleotide sequence. The substantial identity means a nucleotide sequence exhibiting at least 80% of identity, at least 90% of identity, or at least 95% of identity, when the nucleotide sequence of the present disclosure is aligned to match any other sequence as much as possible and the aligned sequences are analyzed with an algorithm commonly used in the art.

According to another aspect of the present disclosure, the present disclosure provides a vector including a nucleic acid molecule encoding the antibody or the antigen-binding fragment thereof of the present disclosure.

In the present specification, the term “vector” refers to a carrier into which a polynucleotide (nucleic acid) sequence can be inserted for introduction into a cell where it can be replicated. The polynucleotide sequence may be exogenous or heterologous. Examples of the vector include, but are not limited to, a plasmid, a cosmid vector and a viral vector (e.g., retrovirus, adenovirus, adeno-associated virus, etc.). Those skilled in the art can construct such vectors through standard recombinant techniques (Maniatis et al., Molecular Cloning, A Laboratory Manual, Cold Spring Harbor Press, Cold Spring Harbor, N.Y., 1988; and Ausubel et al., In: Current Protocols in Molecular Biology, John, Wiley & Sons, Inc., N.Y., 1994, etc.).

In the present specification, the term “expression vector” refers to a vector containing a nucleotide sequence coding for at least a part of a gene product to be transcribed. In some cases, the RNA molecule is then translated into a protein, a polypeptide or a peptide. The expression vector can contain a variety of regulatory sequences. In addition to a regulatory sequence that regulates transcription and translation, the vector and the expression vector may contain nucleic acid sequences that serve other functions as well.

According to another aspect of the present disclosure, the present disclosure provides a cell including the vector.

According to another aspect of the present disclosure, the present disclosure provides a cell transformed with the vector.

In the present specification, the term “cell” includes eukaryotic and prokaryotic cells, and refers to any transformable cell that can replicate the vector or can express a gene encoded by the vector. The cell may be transfected, transduced or transformed by the vector, which means a process in which an exogenous polynucleotide (nucleic acid molecule) is delivered or introduced into the host cell. In the present specification, the term “transformation” is used as a meaning that includes transfection and transduction.

Specifically, the (host) cell of the present disclosure includes but is not limited to an insect cell or a mammalian cell. More specifically, the insect cell may be Sf9 cells, and the mammalian cell may be HEK293 cells, Hela cells, ARPE-19 cells, RPE-1 cells, HepG2 cells, Hep3B cells, Huh-7 cells, C8D1a cells, Neuro2A cells, CHO cells, MES13 cells, BHK-21 cells, COS7 cells, COP5 cells, A549 cells, MCF-7 cells, HC70 cells, HCC1428 cells, BT-549 cells, PC3 cells, LNCaP cells, Capan-1 cells, Panc-1 cells, MIA PaCa-2 cells, SW480 cells, HCT116 cells, LoVo cells, A172 cells, MKN-45 cells, MKN-74 cells, Kato-III cells, NCI-N87 cells, HT-144 cells, SK-MEL-2 cells, SH-SY5Y cells, C6 cells, HT-22 cells, PC-12 cells, NIH3T3 cells, etc.

In addition, the antigen-binding fragment of the anti-B7-H3 antibody of the present disclosure may be used for genetic modification such that an immune cell expresses a chimeric antigen receptor.

In the present specification, the term “immune cell” refers to a cell of the immune system that can be classified as lymphocytes (e.g., T cells, B cells and NK cells), neutrophils, and monocytes/macrophages. In some aspects, the immune cell may be a T cell. In some aspects, the immune cell may be an NK cell.

The immune cell (e.g., T cells) may be modified by one or more methods. The immune cell (e.g., T cells) may express at least one non-natural molecule as a receptor for an antigen existing on the surface of cells of one or more class. In some aspects, the immune cell includes immune cells (e.g., T cells) not found in nature because it is one modified to express at least one synthetic molecule not found in nature. In a specific aspect, the immune cell (e.g., T cells) is modified to express at least one chimeric antigen receptor (CAR), including a CAR targeting a specific tumor antigen such as B7-H3. In a specific aspect, the immune cell may be T cells, e.g., CD4+ T cells, CD8+ T cells, Treg cells, Th1 T cells, Th2 T cells, Th17 T cells, nonspecific T cells, or a population of T cells including any combination thereof. An immune cell (e.g., T cells) modified with a chimeric antigen receptor has a high therapeutic potential for cancer treatment. Using a CAR, a receptor can be programmed to recognize an antigen, which when bound, activates immune cells to kill the cells expressing that antigen. Therefore, immune cells expressing CAR(s) for an antigen expressed on tumor cells can target and kill the tumor cells. For example, recent clinical trials for CD19-targeted CAR-transduced T cells (CD19-CAR T cells) against hematologic malignancies showed a strong effect of CAR T technology (Kochenderfer, J. N. et al. (2010) Blood 116:4099-4102; Porter, D. L., et al. (2011) N. Engl. J. Med. 365:725-733; Grupp, S. A. et al. (2013) N. Engl. J. Med. 368:1509-1518, Kochenderfer, J. N. et al. (2015) J. Clin. Oncol. 33:540-549, Brown, C. E. et al. (2016) N. Engl. J. Med. 375:2561-2569).

The cell of the present disclosure is specifically an isolated cell.

The cell of the present disclosure is specifically an in-vitro cell.

The cell of the present disclosure is specifically an ex-vivo cell.

According to another aspect of the present disclosure, the present disclosure provides a medical use of an ingredient selected from a group consisting of the anti-B7-H3 antibody or an antigen-binding fragment thereof, a nucleic acid encoding the antibody or the antigen-binding fragment thereof, a vector including the nucleic acid, and a cell including the vector or transformed with the vector.

According to another aspect of the present disclosure, the present disclosure provides a composition containing an ingredient selected from a group consisting of the anti-B7-H3 antibody or an antigen-binding fragment thereof, a nucleic acid encoding the antibody or the antigen-binding fragment thereof, a vector including the nucleic acid, and a cell including the vector or transformed with the vector.

According to a specific exemplary embodiment of the present disclosure, the composition of the present disclosure is a pharmaceutical composition for preventing or treating cancer.

According to another aspect of the present disclosure, the present disclosure provides a method for treating cancer, which includes a step of administering an ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector to a subject in need thereof.

The present specification discloses a method for treating cancer (or tumor), which includes administering an ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector to a subject. In a specific exemplary embodiment, the anti-B7-H3 antibody or the antigen-binding fragment thereof binds specifically to B7-H3 and exhibits T cell-mediated cancer cell-killing effect.

In some exemplary embodiments, the method disclosed in the present specification inhibits and/or reduces the growth of cancer (or tumor) in a subject. In a specific exemplary embodiment, tumor growth (for example, in tumor volume or weight) is reduced by at least 5%, at least 10%, at least 20%, at least 30%, at least 40%, at least 50%, at least 60%, at least 70%, at least 80%, at least 90%, or 100% as compared to a reference (for example, tumor volume or weight in a subject not administered with the composition disclosed in the present specification). In some exemplary embodiments, the method disclosed in the present specification increases average tumor growth inhibition (TGI) by at least 5%, at least 10%, at least 20%, at least 30%, at least 40%, at least 50%, at least 60%, at least 70%, at least 80%, at least 90%, or 100% as compared to a reference (for example, frequency in a subject not administered with the composition disclosed in the present specification). In some exemplary embodiments, the administration of the composition disclosed in the present specification (for example, the anti-B7-H3 antibody) increases survival by at least 1 day, 2 days, 3 days, 4 days, 5 days or 6 days on average as compared to a reference (for example, subject not administered with the composition disclosed in the present specification, e.g., the anti-B7-H3 antibody).

In some exemplary embodiments, the tumor that may be treated by the method disclosed in the present specification is derived typically from cancers responsive to existing anticancer agents and cancers not responsive to existing anticancer agents. In some exemplary embodiments, the cancer is one having solid tumors or blood malignancies (liquid tumors).

Non-limiting examples of cancers requiring treatment include brain tumor, medulloblastoma, leptomeningeal metastatic cancer, squamous cell carcinoma, small-cell lung carcinoma (SCLC), non-small-cell lung carcinoma, squamous non-small-cell lung carcinoma (NSCLC), non-squamous NSCLC, gastrointestinal tract cancer, renal cancer (e.g., clear cell carcinoma), ovarian cancer, liver cancer, bile duct cancer, nasopharyngeal cancer, colorectal cancer, endometrial cancer, kidney cancer (e.g., renal cell carcinoma (RCC)), prostate cancer (e.g., hormone-refractory prostate cancer), thyroid cancer, parathyroid cancer, pancreatic cancer, cervical cancer, stomach cancer, bladder cancer, hepatocellular carcinoma, breast cancer, colon cancer, head and neck cancer, laryngeal cancer, germ cell tumor, childhood cancer, nasal NK cell lymphoma, melanoma (e.g., metastatic malignant melanoma, such as cutaneous or intraocular malignant melanoma), bone cancer, skin cancer, uterine cancer, cancer of the anal region, testicular cancer, fallopian tube cancer, carcinoma of the endometrium, carcinoma of the cervix, carcinoma of the vagina, carcinoma of the vulva, esophageal cancer, cancer of the small intestine, colon cancer, mast cell tumor, cancer of the endocrine system, cancer of the parathyroid gland, cancer of the adrenal gland, sarcoma of soft tissue, cancer of the urethra, cancer of the penis, solid tumor of childhood, cancer of the ureter, carcinoma of the renal pelvis, tumor angiogenesis, pituitary adenoma, Kaposi's sarcoma, epidermoid cancer, squamous cell carcinoma, T-cell lymphoma, environmentally induced cancers including those induced by asbestos, virus-related cancers or virus-derived cancers (e.g., human papilloma virus (HPV)-related or derived tumor), hematologic malignancies derived from either of two major blood cell lineages, i.e., the myeloid cell line (which produces granulocytes, erythrocytes, thrombocytes, macrophages and mast cells) or the lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (M0), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyosarcoma; seminoma, teratocarcinoma, tumors of the central and peripheral nervous, including astrocytoma, schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers.

According to an exemplary embodiment of the present disclosure, the cancer may be one or more selected from a group consisting of brain tumor, childhood cancer, medulloblastoma, leptomeningeal metastatic cancer, breast cancer, lung cancer, pancreatic cancer, bowel cancer, liver cancer, prostate cancer, ovarian cancer, stomach cancer, esophageal cancer, lymphoma, melanoma, kidney cancer, fibrosarcoma, colon cancer, colorectal cancer, endometrial cancer, thyroid cancer, parathyroid cancer, cervical cancer, bladder cancer, head and neck cancer, bone cancer, skin cancer, uterine cancer, testicular cancer, bile duct cancer, bronchial cancer, nasopharyngeal cancer, laryngeal cancer and ureteral cancer.

In some exemplary embodiments, the method disclosed in the present specification may be used to treat metastatic cancer, unresectable, refractory cancer (e.g., cancers resistant to existing cancer therapy, such as immunotherapy, e.g., therapy using a blocking PD-(L)1 antibody), and/or recurrent cancer. In a specific exemplary embodiment, the method disclosed in the present specification may be used to treat recurrent cancer.

According to a specific exemplary embodiment of the present disclosure, the pharmaceutical composition may be used in combination with an additional anticancer agent including an immunotherapeutic agent, a chemotherapeutic agent, a targeted therapeutic agent, or a radiotherapeutic agent.

In some exemplary embodiments, the method disclosed in the present specification includes chemotherapy as an existing cancer therapy.

In some exemplary embodiments, the method disclosed in the present specification effectively increases the survival duration of a subject. For example, the survival duration of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, or longer as compared to a reference subject (e.g., a subject not treated with the composition disclosed in the present specification, e.g., the antibody-drug conjugate). In another specific exemplary embodiment, the method disclosed in the present specification increases the survival duration of the subject as compared to the survival duration of a reference subject (e.g., a subject not treated with the composition disclosed in the present specification, e.g., the anti-B7-H3 antibody) (by about 1 month or longer, about 2 months or longer, about 3 months or longer, about 4 months or longer, about 5 months or longer, about 6 months or longer, about 7 months or longer, about 8 months or longer, about 9 months or longer, about 10 months or longer, about 11 months or longer, or about 1 year or longer).

In some exemplary embodiments, the method of the present disclosure increases the duration of a period with no disease progression in a subject. For example, the duration of a period with no disease progression in a subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year as compared to a reference subject (e.g., a subject not treated with the composition disclosed in the present specification, e.g., the anti-B7-H3 antibody).

In some exemplary embodiments, the method disclosed in the present specification increases response rate in a subject. For example, the response rate in the subject is increased by at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 70%, at least about 75%, at least about 80%, at least about 85%, at least about 90%, at least about 95%, at least about 99%, or at least about 100% as compared to a reference subject (e.g., a subject not treated with the composition disclosed in the present specification, e.g., the anti-B7-H3 antibody).

In some exemplary embodiments, the subject to be treated by the method disclosed in the present specification is a non-human animal, e.g., rat or mouse. In some exemplary embodiments, the subject to be treated by the method disclosed in the present specification is human.

The present disclosure also provides a method for treating cancer in a subject in combination with another anticancer agent. In some exemplary embodiments, in the method of the present disclosure, the anti-B7-H3 antibody may be administered in combination with at least one another anticancer agent and/or immunoregulatory agent, e.g., a T-cell-stimulating (e.g., activating) agent as a combination therapy. In some exemplary embodiments, the anti-B7-H3 antibody may be administered in combination with other compounds, drugs and/or agents used in cancer treatment. Such compounds, drugs and/or agents may include, e.g., a chemotherapeutic drug, a small-molecule drug, or an antibody that stimulates immune response. In some exemplary embodiments, the method disclosed in the present specification is used in combination with standard treatment (e.g., surgery, radiation therapy or chemotherapy). In another specific exemplary embodiment, the method disclosed in the present specification is used as a maintenance therapy, e.g., for preventing the onset or recurrence of tumors.

In some exemplary embodiments, the anti-B7-H3 antibody disclosed in the present specification may be used in combination with one or more additional anticancer agent, including an immunotherapeutic agent, a chemotherapeutic agent, a targeted therapeutic agent, a radiotherapeutic agent, or any combination thereof.

The present specification discloses a composition containing the anti-B7-H3 antibody or the antigen-binding fragment thereof of the present specification, with a desired level of purity in a pharmaceutically acceptable carrier, excipient or stabilizer (Remington's Pharmaceutical Sciences (1990) Mack Publishing Co., Easton, PA). The acceptable carrier, excipient or stabilizer is nontoxic to a recipient at the used dose and concentration, and examples thereof include a buffer, e.g., phosphate, citrate and other organic acids; an antioxidant including ascorbic acid and methionine; a preservative (e.g., octadecyl dimethyl benzyl ammonium chloride; hexamethonium chloride; benzalkonium chloride; phenol, butyl or benzyl alcohol; an alkyl paraben, e.g., methyl or propyl paraben; catechol; resorcinol; cyclohexanol; 3-pentanol; and m-cresol); a low-molecular-weight (less than about 10 residues) polypeptide; a protein, e.g., serum albumin, gelatin, or immunoglobulin; a hydrophilic polymer, e.g., polyvinylpyrrolidone; an amino acid, e.g., glycine, glutamine, asparagine, histidine, arginine, or lysine; a monosaccharide, a disaccharide, and other carbohydrates including glucose, mannose, or dextrin; a chelating agent, e.g., EDTA; a sugar, e.g., sucrose, mannitol, trehalose or sorbitol; a salt-forming counter-ion, e.g., sodium; a metal complex (e.g., Zn-protein complex); and/or a non-ionic surfactant, e.g., TWEEN®, PLURONICS® or polyethylene glycol (PEG).

In some exemplary embodiments, the pharmaceutical composition contains an ingredient selected from a group consisting of the anti-B7-H3 antibody or the antigen-binding fragment thereof disclosed in the present specification, a nucleic acid encoding the antibody or the antigen-binding fragment thereof, a vector including the nucleic acid and a cell including the vector or transformed with the vector in the pharmaceutically acceptable carrier. In a specific exemplary embodiment, the pharmaceutical composition contains an effective amount of the anti-B7-H3 antibody disclosed in the present specification and, optionally, one or more preventive or therapeutic agent in the pharmaceutically acceptable carrier. In some exemplary embodiments, the ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector is the only active ingredient contained in the pharmaceutical composition.

Pharmaceutically acceptable carriers used in parenteral preparations include an aqueous vehicle, a nonaqueous vehicle, an antimicrobial agent, an isotonic agent, a buffer, an antioxidant, a local anesthetic, a suspending or dispersing agent, an emulsifying agent, a metal ion sequestering or chelating agent, and other pharmaceutically acceptable materials. Examples of the aqueous vehicle include sodium chloride injection, Ringer's injection, isotonic dextrose injection, sterile water injection, dextrose, and lactated Ringer's injection. The nonaqueous vehicle includes plant-derived fixed oil, cottonseed oil, corn oil, sesame oil, and peanut oil. An antimicrobial agent of bacteriostatic or fungistatic concentration can be added to a parenteral preparation packaged in a multi-dose container, and it may include phenol, cresol, a mercurial, benzyl alcohol, chlorobutanol, methyl or propyl p-hydroxybenzoate ester, thimerosal, benzalkonium chloride and benzethonium chloride. The isotonic agent includes sodium chloride and dextrose. The buffer includes phosphate and citrate. The antioxidant includes sodium bisulfate. The local anesthetic includes procaine hydrochloride. The suspending or dispersing agent includes sodium carboxymethyl cellulose, hydroxypropyl methylcellulose and polyvinylpyrrolidone. The emulsifying agent includes polysorbate 80 (TWEEN® 80). The metal ion sequestering or chelating agent includes EDTA. The pharmaceutically acceptable carrier includes ethyl alcohol, polyethylene glycol and propylene glycol for water-miscible vehicles, as well as sodium hydroxide, hydrochloric acid, citric acid or lactic acid for control of pH.

The pharmaceutical composition may be formulated depending on administration routes for the subject. Specific examples of the administration route include intranasal, oral, parenteral, intraspinal, intraventricular, pulmonary, subcutaneous or intracardiac routes. Parenteral administration characterized by subcutaneous, intramuscular or intravenous injection is also considered. Injectables can be prepared into conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical composition to be administered may also contain a small amount of nontoxic auxiliary substances such as wetting or emulsifying agents, pH-buffering agents, stabilizers, solubility enhancers and other agents, e.g., sodium acetate, sorbitan monolaurate, triethanolamine oleate, or cyclodextrin.

The anti-B7-H3 antibody preparation for parenteral administration includes a sterile solution that can be injected readily, a sterile, dried, soluble product that can be combined with a solvent immediately before use, e.g., freeze-dried powder, a sterile suspension that can be injected readily, a sterile, dried, soluble product that can be combined with a vehicle immediately before use, and a sterile emulsion. The solution may be aqueous or nonaqueous.

The anti-B7-H3 antibody disclosed in the present specification may also be formulated to target specific tissues, receptors, or other body parts of the subject to be treated. Various targeting methods are known to those skilled in the art. The use of the targeting methods is considered for the composition of the present disclosure. For non-limiting examples of the targeting methods, see U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The composition used for administration into the body may be sterilized. For example, it may be sterilized by filtration through a sterile filtration membrane.

According to another aspect, the present disclosure provides a composition for diagnosing cancer, which contains an ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector.

According to another aspect, the present disclosure provides a kit for diagnosing cancer, which includes an ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector.

The kit may include an instruction, and the instruction may include a method for using the diagnostic kit, which includes a step of treating a sample (body fluid, blood, urine, cell, tissue, etc.) isolated from a subject in need of diagnosis with the anti-B7-H3 antibody or the antigen-binding fragment thereof, nucleic acid encoding the antibody or the antigen-binding fragment thereof, a vector including the nucleic acid or a cell including the vector or transformed with the vector.

In some exemplary embodiments, the subject in need of diagnosis is a non-human animal, e.g., rat, mouse, etc. In some exemplary embodiments, the subject to be diagnosed is human.

According to another aspect, the present disclosure provides a method for providing information for diagnosis of cancer, which includes a step of treating a sample isolated from a subject in need of diagnosis with an ingredient selected from a group consisting of the anti-B7-H3 antibody or the antigen-binding fragment thereof, nucleic acid encoding the antibody or the antigen-binding fragment thereof, a vector including the nucleic acid and a cell including the vector or transformed with the vector.

### Advantageous Effects

The features and advantages of the present disclosure may be summarized as follows:

(i) The present disclosure provides an anti-B7-H3 antibody or an antigen-binding fragment thereof, which binds to a specific epitope located within the amino acid sequence of a domain of B7-H3.

(ii) In addition, the present disclosure provides a pharmaceutical composition for preventing or treating cancer, which contains an ingredient selected from a group consisting of the anti-B7-H3 antibody or the antigen-binding fragment thereof, a nucleic acid encoding the antibody or the antigen-binding fragment thereof, a vector including the nucleic acid and a cell including the vector or transformed with the vector, and a pharmaceutically acceptable carrier.

(iii) The antibody specifically binds to various types of cancer cell lines expressing B7-H3, demonstrating T cell-mediated cancer cell-killing effect, and binds to epitopes in both humans and mice, thus allowing for effective evaluation of anticancer effect in preclinical trials when developed into a therapeutic antibody.

### BRIEF DESCRIPTION OF DRAWINGS

FIG. 1 schematically shows biopanning using an antibody library.

FIG. 2a shows a result of investigating the binding ability of recombinant human 4lg B7-H3, recombinant human 2lg B7-H3 and recombinant mouse B7-H3 of 2D IgG, constructed as complete antibodies, by indirect ELISA.

FIG. 2b shows a result of investigating the binding ability of 2D IgG for Raji and BT-20 cells by FACS.

FIG. 3a shows a result of investigating the binding ability of recombinant human 4lg B7-H3, recombinant human 2lg B7-H3 and recombinant mouse B7-H3 of G2D IgG, which are humanized antibodies, by indirect ELISA, and FIG. 3b shows a result of investigating the binding ability of recombinant human 4lg B7-H3, recombinant human 2lg B7-H3 and recombinant mouse B7-H3 of G2D IgG, which are humanized antibodies, by SPR.

FIG. 4 shows the interaction between the B7 family proteins expressed on antigen-presenting cells (APCs) including cancer cells and T cells.

FIG. 5 shows a result of investigating the specificity of the B7 family proteins of G2D IgG for B7-H3 by indirect ELISA.

FIGS. 6a and 6b show a result of investigating the binding ability of G2D IgG for B7-H3-expressing Raji cells by FACS. FIG. 6a shows a result of investigating the binding ability for human 4lg, 2lg B7-H3-expressing Raji cells, and FIG. 6b shows a result of investigating binding ability for mouse B7-H3-expressing Raji cells.

FIGS. 7a to 7c show a result of investigating the binding ability of G2D IgG for 16 types of human cancer cells by FACS. FIG. 7a shows a result of investigating binding ability for colon cancer, liver cancer, prostate cancer and ovarian cancer cells; FIG. 7b for brain tumor and breast cancer cells; and FIG. 7c for pancreatic cancer and lung cancer cells.

FIG. 8 shows a result of investigating the binding ability of G2D IgG for 4 types of mouse cancer cells confirmed to express B7-H3 by FACS.

FIG. 9 shows a result of investigating the immunogenicity of G2D IgG in silico.

FIGS. 10a to 10e show a result of investigating the epitopes of G2D IgG. FIG. 10a shows a result of investigating the binding of G2D IgG for the recombinant 4lg B7-H3 protein by western blot in order to confirm the epitope class of G2D IgG, and FIG. 10b shows a result of confirming the human 4lg B7-H3 peptide to which G2D IgG binds. FIG. 10c shows a result of investigating the binding of G2D IgG for the human 4lg B7-H3 peptide including EVQVP, which is expected as an epitope of G2D IgG, and FIG. 10d displays the epitope of G2D IgG in the entire amino acids of human 4lg B7-H3. FIG. 10e displays the epitope of G2D IgG in the human 4lg B7-H3 protein.

FIGS. 11a and 11b show a result of investigating the anticancer efficacy of G2D IgG in syngeneic mice transplanted with GL261 cells. FIG. 11a shows a result of measuring tumor volume, and FIG. 11b shows a result of measuring body weight.

### BEST MODE

Hereinafter, the present disclosure will be described in more detail through examples. The examples are intended only to describe the present disclosure more specifically, and it will be obvious to those having ordinary knowledge in the art that the scope of the present disclosure is not limited by the examples.

### EXAMPLES

### Example 1: Screening of B7-H3-Positive Clones

### 1-1. Screening of Clones Binding to B7-H3

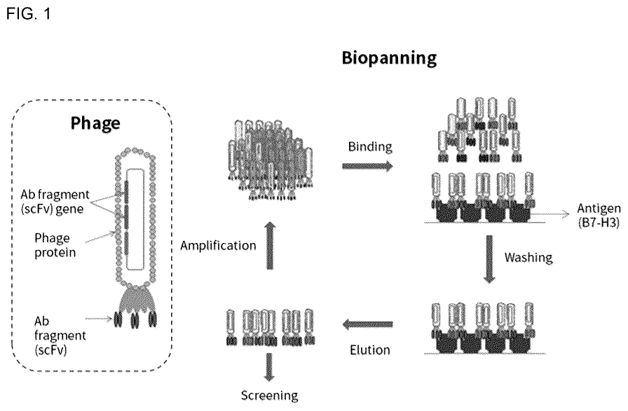

B7-H3-positive clones were screened using a phage display technology as shown in FIG. 1. First, 2.5 μg of recombinant human 4lg B7-H3 (Sino Biological, China) was conjugated to magnetic beads (Thermo Fisher Scientific, USA). After removing phages binding to Raji cells in advance by reacting with Raji cell pellets known to not express B7-H3, phages having affinity for B7-H3 were bound by reacting a chicken naive phage library dispersed in a 3% bovine serum albumin (Millipore, USA) solution with the 4lg B7-H3 conjugated to the magnetic beads at room temperature for 2 hours, washed with a buffer solution containing 0.5% Tween 20 (Amresco, USA), and then eluted using an acidic solution. For the next round of biopanning, the eluted phages were proliferated by infecting with E. coli ER2738 and incubating overnight. Biopanning was conducted by repeating this procedure 4 times. Phages with high binding ability were accumulated by increasing the number of washing from 1 in the first washing to 3 in the second and third washing and 5 in the fourth washing.

96 clones screened from the plate of the products of the fourth biopanning were incubated in a 96-deep well plate overnight at 37° C. after adding 100 μg/mL carbenicillin, 70 μg/mL kanamycin and VCSM13 helper phage to induce the proliferation of phages expressing scFv clones. The obtained culture was centrifuged to obtain a supernatant containing phages, which was reacted at 37° C. for 2 hours after adding to an ELISA plate coated with recombinant human 4lg B7-H3, recombinant human 2lg B7-H3 (R&D Systems, USA) and recombinant mouse B7-H3 (Sino Biological, China), respectively, and the clones binding to B7-H3 were identified by phage ELISA using HRP-conjugated anti-M13 antibody (Sino Biological, China) as a secondary antibody. Then, 2D clones exhibiting strong signals were selected.

### 1-2. Sequencing of Screened Antibodies

ER2738 having the screened 2D clones exhibiting positive signals for B7-H3 was incubated overnight using an SB medium. After acquiring plasmid DNAs using a DNA mini prep kit (Geneall, Korea), base sequence was analyzed using the primers of Table 1. The analyzed 2D clones had CDR sequences of Table 2. The light chain and heavy chain variable region amino acid sequences of the 2D clones are shown in Table 3.

TABLE 1
Primers used for sequencing of screened clones
scFv sequencing primer

| | | |
|---|---|---|
| 1 | ACA CTT TAT GCT TCC GGC | Forward |
| | | (SEQ ID NO: 1 |
| 2 | CAA AAT CAC CGG AAC CAG AG | Reverse |
| | | (SEQ ID NO: 2 |

TABLE 2
CDR amino acid sequences of screened 2D clones
Light chain variable region CDRs

| LCDR1 | LCDR2 | LCDR3 |
|---|---|---|
| SGGSIGYG | YNDRRPS | GSADSSSTYTGI |
| (SEQ ID NO: 3) | (SEQ ID NO: 4) | (SEQ ID NO: 5) |

Heavy chain variable region CDRs

| HCDR1 | HCDR2 | HCDR3 |
|---|---|---|
| SYPMV | SINSGGSWTGYGAAVKG | AYGAATIDA |
| (SEQ ID NO: 6) | (SEQ ID NO: 7) | (SEQ ID NO: 8) |

TABLE 3
Amino acid sequences of light chain and heavy
chain variable regions of screened clones

| | | SEQ |
|---|---|---|
| | Amino acids | ID NO |
| Light chain | ALTQPSSVSANLGGTVKITCSGGSIGY | 9 |
| variable | GWYQQKAPGSAPVTVIYYNDRRPSDIP | |
| region | SRFSGSKSGSANTLTITGVQADDEAIY | |
| | YCGSADSSSTYTGIFGAGTTLTVL | |
| Heavy chain | AVTLDESGGGLQTPGGGLSLVCKASGF | 10 |
| variable | DFSSYPMVWVRQAPGKGLEYVASINSG | |
| region | GSWTGYGAAVKGRATISRDNGQSTVRL | |
| | QLNNLRAEDTATYYCARAYGAATIDAW | |
| | GHGTEVIVSS | |

### Example 2: Conversion of Screened Clones to Complete Antibodies and Expression/Purification

### 2-1. Conversion of Screened Clones to Complete Antibodies

Since the 2D clones screened in Example 1 are in the form of scFv, they were converted to complete antibodies (full-length IgG) through PCR. First, the fragments of the variable region and constant region of heavy chain and light chain were acquired from pComb3x containing scFv by PCR using the combinations of V_{L}, C_{K}, V_{H} and C_{H} primers of Table 4. Then, the heavy chain and light chain of the antibodies were secured by PCR using the combinations of LC and HC primers of Table 4. The heavy chain was treated with EcoRI and NotI enzymes (New England Biolab, UK) and ligated to a pCMV vector (Thermo Fisher Scientific, USA), which is an expression vector for animal cells treated with the same restriction enzymes. In addition, the light chain was treated with an XbaI enzyme (New England Biolab, UK) and ligated to a pCMV vector treated with the same restriction enzyme. DH5a competent cells (New England Biolab, UK) were transformed by applying heat shock using the ligated plasmids, and the acquired colonies were mass-cultured.

TABLE 4
Primers used for cloning of G2D complete antibody

| | Primers | Sequences | SEQ ID NO |
|---|---|---|---|
| V_{L} | Forward | GGT CTT TGT ATA CAT GTT GCT GTG GTT GTC TGG TGT TGA AGG AGC CCT GAC | 11 |
| | | TCA GCC GT | |
| | Reverse | GGC CAC GGT CCG TAG GAC GGT CAG GGT | 12 |
| C₈ | Forward | CGG ACC GTG GCC GCC CCC TC | 13 |
| | Reverse | TAG TTC TAG AAC TAG CAC TCG CCC CG | 14 |
| LC | Forward | GGG AAT TCT AGA GGA TCG AAC CCT TTG CAA GCT TCG GCA CGA GCA GAC CAG | 15 |
| | | CAT GGG CAT CAA GAT GGA GAC ACA TTC TCA GGT CTT TGT ATA CAT GTT G | |
| | Reverse | TAG TTC TAG AAC TAG CAC TCG CCC CG | 14 |
| V_{H} | Forward | CTT CCT GTC AGT AAC TAC AGG TGT CCA CTC CGC CGT GAC GTT GGA C | 16 |
| | Reverse | CCC TTG GTG GAG GCG GAG GAG ACG ATG AC | 17 |
| C_{H} | Forward | GCC TCC ACC AAG GGC CC | 18 |
| | Reverse | GTG AGC GGC CGC TCA CTT GCC GGG GGA | 19 |
| HC | Forward | CAG AAT TCA CTC TAA CCA TGG AAT GGA GCT GGG TCT TTC TCT TCT TCC TGT | 20 |
| | | CAG TAA CTA CAG | |
| | Reverse | GTG AGC GGC CGC TCA CTT GCC GGG GGA | 19 |

### 2-2. Expression/Purification

The plasmids of the heavy chain and light chain of the complete antibodies were transduced into Expi293F cells (Invitrogen, USA) using polyethylenimine (PEI) (Polysciences, USA) and 150 mM NaCl, and suspension-cultured in an Erlenmeyer flask for 7 days using a Freestyle 293 expression medium (Invitrogen, USA) under the condition of 37° C., 8% CO₂ and 55% humidity. The cell culture was centrifuged at 4,000 rpm for 10 minutes, and the supernatant was filtered through a 0.22-μm filter. The filtered supernatant was induced to bind to a 1-mL HiTrap Mabselect PrismA column (GE Healthcare, USA) at 4° C. After washing the bound resin with 10 cv (column volume) of 20 mM sodium phosphate (pH 7.0) and 1 M sodium chloride solutions, the bound antibodies were eluted using 100 mM sodium citrate (pH 3.0) and 150 mM sodium chloride solutions and then neutralized with 1 M Tris-HCL (pH 9.0). After buffer exchange with pH 7.2-7.4 PBS using a Slide-A-Lyzer dialysis cassette (Thermo Fisher Scientific, USA), the size of the light chain and heavy chain and purity of the purified antibodies were identified by SDS-PAGE. As a result, it was confirmed that the antibodies with a molecular weight matching with the theoretical calculation were produced with high purity.

### Example 3: Analysis of Antigen-Binding Ability

### 3-1. Analysis of Binding Ability for Recombinant Antigen Protein

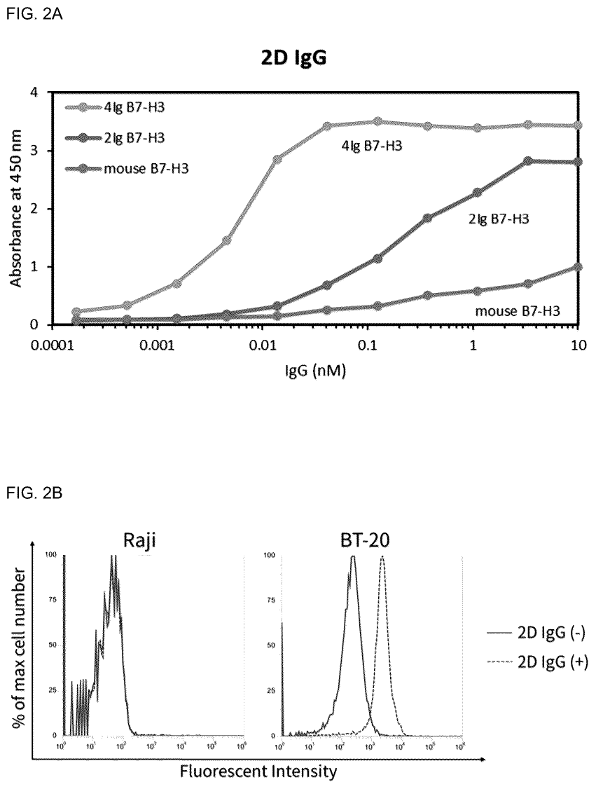

The binding ability of 2D IgG prepared in Example 2 for recombinant B7-H3 protein was investigated by indirect ELISA. For indirect ELISA, recombinant human 4lg B7-H3, recombinant human 2lg B7-H3 and recombinant mouse B7-H3 were diluted respectively to 1 μg/mL in 50 μL of PBS and adsorbed overnight onto a 96-well immunoplate (Corning, USA) by storing at 4° C. After reaction with a buffer solution containing 3% bovine serum albumin at room temperature for 1 hour, followed by washing 3 times with a buffer solution containing 0.5% Tween 20, each well was treated with 50 μL of the antibodies diluted serially (0.0001, 0.0003, 0.001, 0.003, 0.01, 0.03, 0.1, 0.3, 1, 3 and 10 nM). For antigen-antibody binding, the mixture was reacted at room temperature for 2 hours, and then washed 3 times with a buffer solution containing 0.5% Tween 20. After treating each well with 50 μL of anti-human immunoglobulin Fc-HRP antibodies (Jackson Immunoresearch, USA) diluted to 1:3,000, reaction was conducted at room temperature for 1 hour. After the reaction was completed, followed by washing 3 times with a buffer solution containing 0.5% Tween 20, 50 μL of 3,3′,5,5′-tetramethylbenzidine (TMB) (Thermo Fisher Scientific, USA) was added and color development was conducted for 10 minutes. As a result of measuring absorbance at 450 nm using a spectrophotometer (Biotek, USA), it was confirmed that 2D IgG binds to B7-H3, and the binding ability was in the order of 4lg B7-H3, 2lg B7-H3 and mouse B7-H3 (FIG. 2a).

### 3-2. Analysis of Binding Ability for B7-H3-Expressing Cells

Raji cells are known as B7-H3-negative cells not expressing B7-H3 (Mol Ther Oncolytics. 2020; 17:180-189), and it is known that B7-H3 is overexpressed in various breast cancer cells including BT-20 cells (Mol Cancer Ther. 2011; 10(6): 960-71).

The binding ability of 2D IgG was analyzed by FACS using B7-H3-negative Raji cells and B7-H3-positive BT-20 cells. 5×10⁵ BT-20 and Raji cells were suspended in PBS containing or not containing 1 μg of 2D IgG. Then, after reaction for 30 minutes at 4° C., the supernatant was removed through centrifugation. After the supernatant was removed, the cells were washed 2 times with PBS. The washed cells were centrifuged at 4° C. for 3 minutes after adding FITC-conjugated Alexa Fluor goat anti-human IgG (H+L) antibodies (Invitrogen, USA). After removing the supernatant, the cells were washed 2 times with PBS and then flow cytometry was conducted. The result is shown in FIG. 2b. 2D IgG did not bind to the B7-H3-negative Raji cells, but bound strongly to the B7-H3-positive BT-20 cells (FIG. 2b).

### Example 4: Preparation of Humanized G2D Antibody

### 4-1. Humanization of 2D

Since 2D IgG is a chicken/human chimeric antibody, it was humanized as follows.

For humanization of 2D, the three FRs (LFR1, LFR2 and LFR3) in the light chain variable region of 2D were substituted with the FRs of IGLV3-25*02, and the three FRs (HFR1, HFR2 and HFR3) in the heavy chain variable region were substituted with the FRs of IGHV3-64*04. Then, in consideration of the similarity or dissimilarity of the physicochemical properties of amino acids, specific amino acids were substituted again with the amino acids of the parent antibody (2D). The amino acids substituted again were L46T, S66K and V71N in the light chain variable region, and T28D and F67A in the heavy chain variable region. The amino acid residues in the antibody domains were designated according to the Kabat EU numbering system (Kabat et al., “Sequences of Proteins of Immunological Interest”, 5th Ed., U.S. Department of Health and Human Services, NIH Publication No. 91-3242, 1991) commonly used in the art. The J gene of the light chain variable region was fixed as FGGGTKLTVL with reference to US2010-0056386, and the J gene of the heavy chain variable region was fixed as WGQGTTVTVSS with reference to US2014-0206849. The humanized 2D was named G2D, and its base sequence is shown in Table 5.

TABLE 5
Light chain and heavy chain variable region
amino acid sequences of G2D
Light chain variable region

| | | |
|---|---|---|
| G2D | SYELTQPPSVSVSPGQTARITCSGGSIGYGWYQQK | SEQ ID |
| (V_{L}) | APGQAPVTVIYYNDRRPSGIPERFSGSKSGTTNTL | NO: 21 |
| | TISGVQAEDEADYYCGSADSSSTYTGIFGGGTKLT | |
| | VL | |

Heavy chain variable region

| | | |
|---|---|---|
| G2D | QVQLVESGGGLVQPGGSLRLSCSASGFDFSSYPMV | SEQ ID |
| (V_{H}) | WVRQAPGKGLEYVSSINSGGSWTGYGAAVKGRATI | |
| | SRDNSKNTLYLQMNSLRAEDTATYYCARAYGAATI | NO: 22 |
| | DAWGQGTTVTVSS | |

In the above table, the underline indicates the CDR regions, and the bold type indicates the amino acids substituted again with those of the parent antibody (2D).

### 4-2. Conversion of G2D to Complete Antibody and Expression/Purification

The DNA of the G2D variable region was synthesized as scFv (Cosmogenetech, Korea) and then converted to a complete antibody by PCR using the primer combinations of Table 6. The conversion to complete antibody and expression/purification were conducted in the same manner as in Example 2.

TABLE 6
Primers used for cloning of G2D complete antibody

| | Primers | Sequences | SEQ ID NO |
|---|---|---|---|
| V_{L} | Forward | TCT CAG GTC TTT GTA TAC ATG TTG CTG TGG TTG TCT GGT GTT GAA GGA TCC | 23 |
| | | TAC GAG TTG ACG CAG | |
| | Reverse | GGG CGG CCA CGG TCC GGA GAA CTG TCA GTT TCG TTC C | 24 |
| C₈ | Forward | CGG ACC GTG GCC GCC CCC TC | 25 |
| | Reverse | TAG TTC TAG AAC TAG CAC TCG CCC CG | 26 |
| LC | Forward | GGG AAT TCT AGA GGA TCG AAC CCT TTG CAA GCT TCG GCA CGA GCA GAC CAG | 27 |
| | | CAT GGG CAT CAA GAT GGA GAC ACA TTC TCA GGT CTT TGT ATA CAT GTT G | |
| | Reverse | TAG TTC TAG AAC TAG CAC TCG CCC CG | 26 |
| V_{H} | Forward | CTT CCT GTC AGT AAC TAC AGG TGT CCA CTC CCA GGT CCA GCT GGT TGA AAG | 28 |
| | Reverse | GGC CCT TGG TGG AGG CGC TAG ACA CTG TCA CGG TTG | 20 |
| C_{H} | Forward | GCC TCC ACC AAG GGC CC | 80 |
| | Reverse | GTG AGC GGC CGC TCA CTT GCC GGG GGA | 31 |
| HC | Forward | CAG AAT TCA CTC TAA CCA TGG AAT GGA GCT GGG TCT TTC TCT TCT TCC TGT | 32 |
| | | CAG TAA CTA CAG | |
| | Reverse | GTG AGC GGC CGC TCA CTT GCC GGG GGA | 31 |

### Example 5: Analysis of Antigen-Binding Ability and Specificity of G2D IgG

### 5-1. Analysis of Binding Ability for Recombinant Antigen Protein by Indirect ELISA

The binding ability of G2D IgG prepared in Example 4 for recombinant B7-H3 protein was investigated by indirect ELISA. The Indirect ELISA was conducted in the same manner as in Example 3-1.

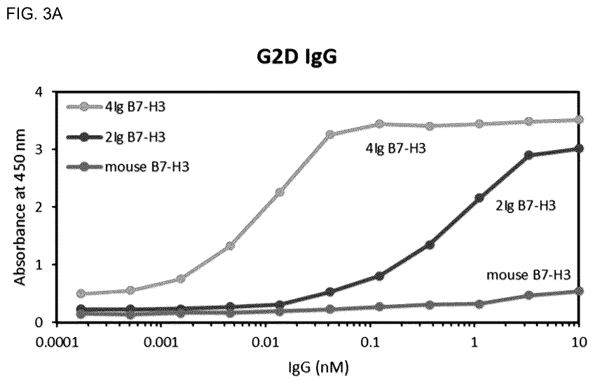

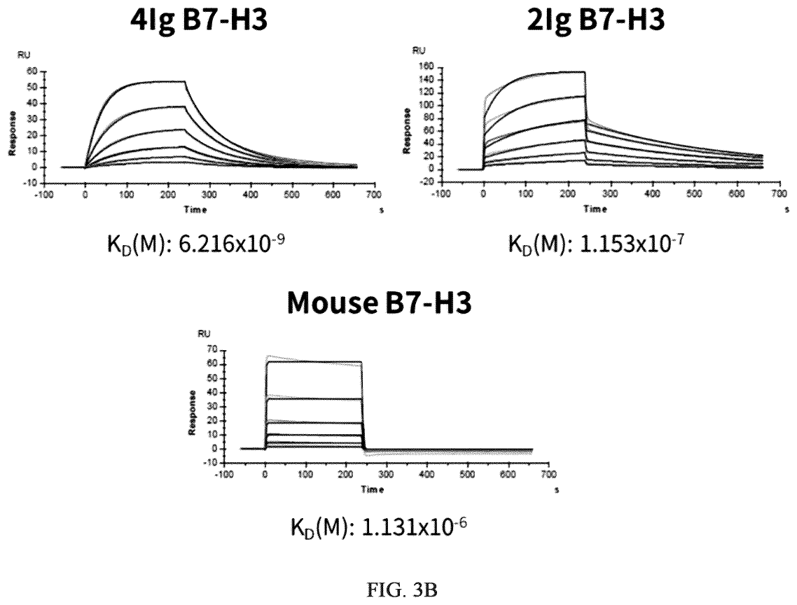

As a result of the indirect ELISA, G2D IgG bound to B7-H3 like 2D IgG, and the binding ability was in the order of 4lg B7-H3, 2lg B7-H3 and mouse B7-H3 (FIG. 3).

### 5-2. Analysis of Binding Ability for Recombinant Antigen Protein by Surface Plasmon Resonance (SPR) Measurement

The binding ability of G2D IgG for B7-H3 was investigated by SPR, which allows the measurement of antigen-antibody binding ability and kinetics based on the optical principle. Since association rate (Kₐ) and dissociation rate (K_{d}) can be determined from the SPR analysis and K_{D} can be calculated therefrom, the quantitative analysis of binding ability is possible.

The SPR analysis was performed using the Biacore T200 instrument (Cytiva, USA). Anti-human Fc antibody (Jackson Immunoresearch, USA) was immobilized on a CM5 chip (Cytiva, USA) with 7658-7908 RU, and then G2D IgG was captured with 400-460 RU. 4lg B7-H3 was injected at 0.3125-10 nM, 2lg B7-H3 at 25-800 nM, and mouse B7-H3 at 15.625-500 nM. Association/dissociation time for B7-H3 was set to 4/7 minutes, respectively. As a result of the analysis, the binding ability for 4lg B7-H3 was at the level of 10⁻⁹ M, the binding ability for 2lg B7-H3 was at the level of 10⁻⁷ M, and the binding ability for mouse B7-H3 was at the level of 10⁻⁶ M (FIG. 3b, Table 7). The result was similar to that of indirect ELISA, and it was confirmed that G2D IgG can bind to all of recombinant 4lg, 2lg and mouse B7-H3.

TABLE 7
Binding ability of G2D IgG for B7-H3 confirmed by SPR analysis

| Analyte | Ligand | Kₐ (1/Ms) | K_{d} (1/s) | K_{D} (M) |
|---|---|---|---|---|
| 4Ig B7-H3 | G2D IgG | 2.357 × 10⁶ | 1.465 × 10⁻² | 6.216 × 10⁻⁹ |
| 2Ig B7-H3 | | 2.494 × 10⁴ | 2.875 × 10⁻³ | 1.153 × 10⁻⁷ |
| Mouse B7-H3 | | 5.270 × 10⁵ | 5.961 × 10⁻¹ | 1.131 × 10⁻⁶ |

### 5-3. Analysis of Specificity for Recombinant B7-Family Proteins

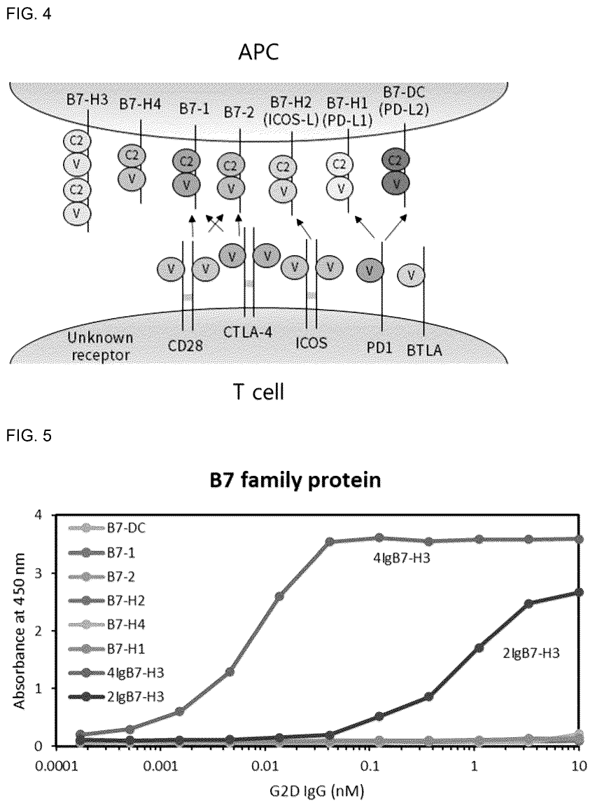

B7-family proteins are expressed on the surface of antigen-presenting cells (APCs) including cancer cells and they regulate the action of T cells through interaction with the proteins on the surface of the T cells (FIG. 4). B7-H3, which is a member of the B7 family proteins expressed on APCs, is known as an immune checkpoint that inhibits the action of T cells although the T cell receptors have not been elucidated yet (Front Immunol. 2021; 12:701006). Since the B7 family proteins regulate the action of T cells through different mechanisms, the specificity of an antibody targeting B7-H3 for B7-H3 is of great importance. Therefore, the specificity of G2D IgG for B7-family proteins was investigated by indirect ELISA.

Indirect ELISA was conducted in the same manner as in Example 3-1 using a 96-well immunoplate coated with B7-DC (Sino Biological, China), B7-1 (Sino Biological, China), B7-2 (Sino Biological, China), B7-H2 (Sino Biological, China), B7-H4 (Sino Biological, China), B7-H1 (Sino Biological, China) and 4lg B7-H3, 2lg B7-H3 at 1 μg/mL.

As a result of the indirect ELISA, G2D IgG bound only to 4lg. 2lg B7-H3 among the B7 family proteins (FIG. 5). This result confirms the superior specificity of G2D IgG for B7-H3.

### 5-4. Analysis of Binding Ability for B7-H3 Expressed on Cell Surface

Although recombinant proteins have the same sequences as cell surface proteins, there may be structural difference. Accordingly, it is essential to verify the binding of an antibody to cell surface proteins. In order to verify binding to B7-H3 expressed on the cell surface, structures including 4lg, 2lg and mouse B7-H3 extracellular domains were introduced into B7-H3-negative Raji cells. The expression of 2lg, 4lg and mouse B7-H3 on the cell surface was confirmed by inserting green fluorescent protein (GFP). The binding of G2D IgG to B7-H3 expressed on the cell surface was verified in the same manner as in Example 3-2. A goat anti-Human lgG (H+L) 594 antibody (Invitrogen, USA) was used as a secondary antibody. Because it is difficult to quantitatively verify the binding to cells, enoblituzumab, which is currently on the clinical phase and patented in Korea (Registration No.: 10⁻¹⁸²⁸⁵⁷⁰), was used as a control antibody.

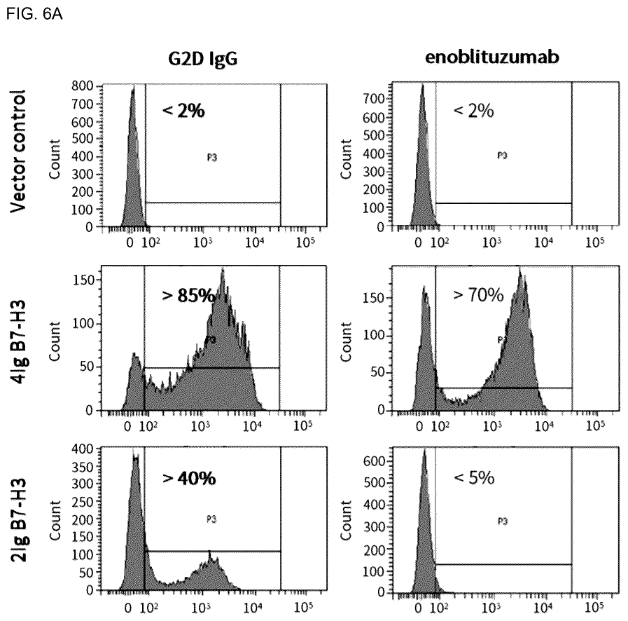

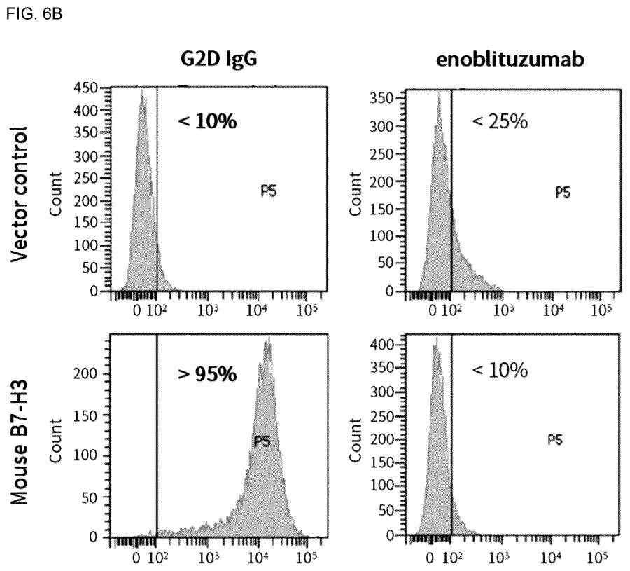

G2D IgG did not bind to Raji-Vector control, wherein B7-H3 was not expressed, but bound to Raji-4lg B7-H3 and Raji-2lg B7-H3, wherein 4lg, 2lg B7-H3 was expressed (FIG. 6a). The binding ability of G2D IgG for 4lg and 2lg B7-H3 expressed on the cell surface was higher than that of the control antibody enoblituzumab. Similarly to human B7-H3, G2D IgG did not bind to Raji-Vector control, but bound to Raji-mouse B7-H3, wherein mouse B7-H3 was expressed (FIG. 6b). The control antibody enoblituzumab did not bind to mouse B7-H3. Since a therapeutic antibody exhibits therapeutic effect by specifically binding to antigens expressed in cells when injected into the body, superior therapeutic effect may be expected as the binding ability for antigens expressed in cells is higher. From FIGS. 6a and b, it can be confirmed that G2D IgG binds with high specificity to cell surface B7-H3.

### 5-5. Analysis of Binding Ability for Human Cancer Cells

Since B7-H3 is expressed in various types of cancers, the binding of G2D IgG to various types of human cancer cells was investigated. The binding ability of G2D IgG for HCT116 and HT29 colon cancer cells; HepG2 and HepG2K liver cancer cells; PC-3 prostate cancer cells; A2780 ovarian cancer cells; A172, U251 and U87 brain tumor cells; MDA-MB-231, SK-BR-3 and MDA-MB-453 breast cancer cells; MIA-PaCa2 and Panc-1 pancreatic cancer cells; and A549 and NCI-H1975 lung cancer cells was investigated according to the method of Example 3-2.

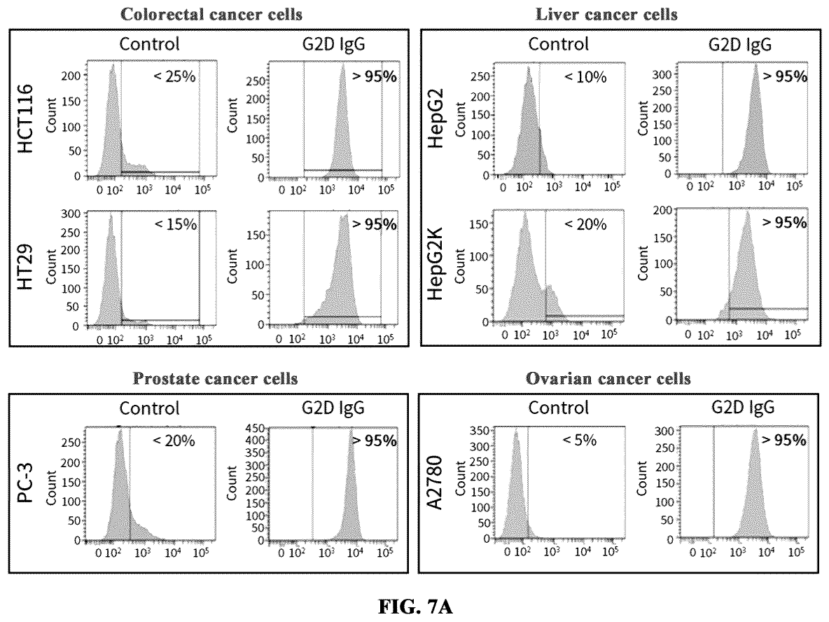

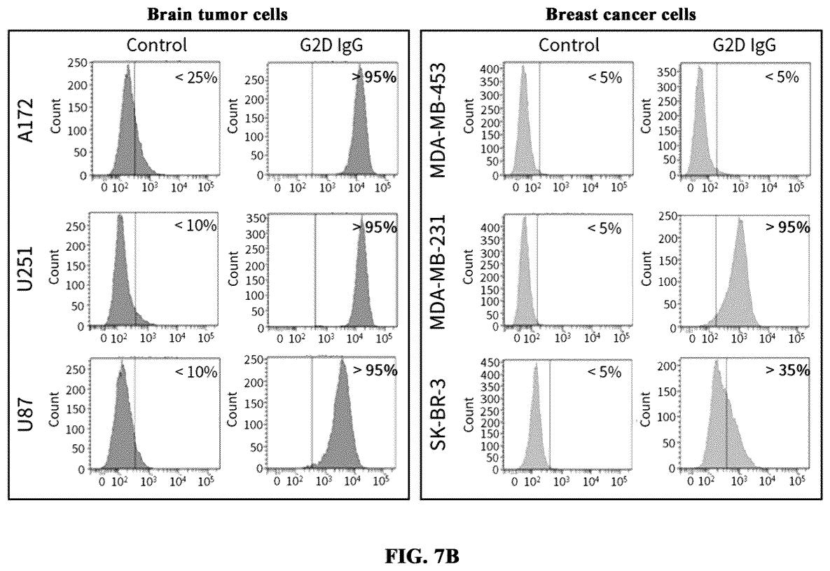

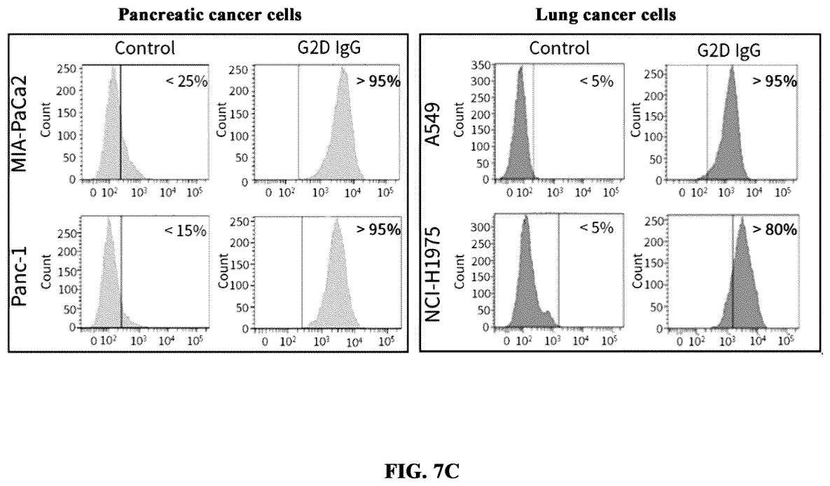

Among the 16 types of cells used, G2D IgG showed distinct peak shift as compared to the control group to which only the secondary antibody was bound, except for the MDA-MB-453 breast cancer cells (FIG. 7). This result means that G2D IgG binds to 15 out of the 16 types of cells used and G2D IgG can bind to cancer cells that express B7-H3 regardless of the type of cancer.

### 5-6. Analysis of Binding Ability for B7-H3-Expressing Mouse Cancer Cells

Antibodies that cross-bind to human and mouse antigens are advantageous for development of therapeutic agents since anticancer effect and the activity of immune cells can be confirmed in a syngeneic mouse model in which the immune system is conserved. In addition, it is advantageous in that toxicity in mouse, which is a small animal, can be confirmed prior to a toxicity test for large animals. Therefore, the binding ability of G2D IgG for 4 types of mouse cancer cells, 3LL (mouse lung cancer cells), 4T1 (mouse breast cancer cells), Panc-02 (mouse pancreatic cancer cells) and GL-261 (mouse brain tumor cells), wherein the expression of B7-H3 was verified, was investigated according to the method of Example 3-2.

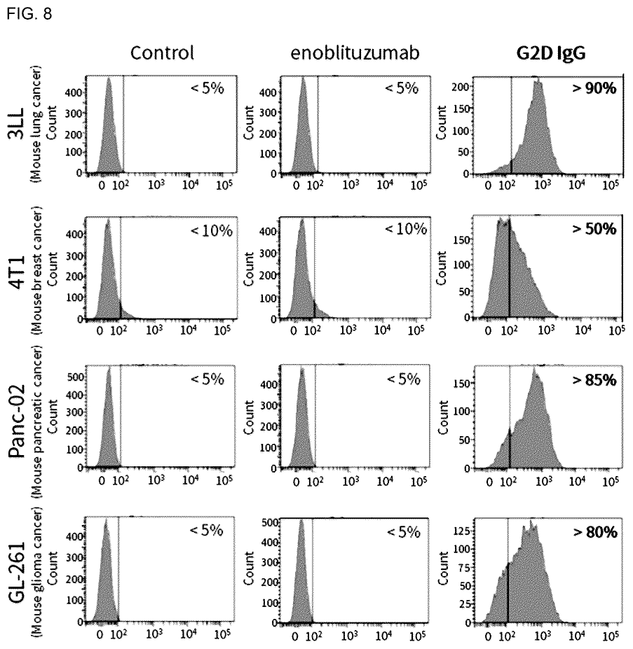

Unlike the control antibody enoblituzumab, G2D IgG bound to all of the 4 types of mouse cancer cells used (FIG. 8). This result means that G2D IgG can be applied to various mouse models including the syngeneic mouse model, and effective evaluation of anticancer effect is possible in preclinical trials when G2D IgG is developed as therapeutic antibody.

### Example 6: Confirmation of Immunogenicity of G2D IgG in Silico

Immunogenicity refers to the ability of a therapeutic antibody which provokes an immune response when it is introduced into the body. The immune response induced by the immunogenicity of a therapeutic antibody may cause severe problems not only in therapeutic efficacy but also in the safety of a patient. Accordingly, it is important to verify the immunogenicity of a therapeutic antibody in drug development. Therefore, the immunogenicity of G2D IgG was investigated by an in-silico method.

Since the probability of immunogenicity increases when an antibody protein binds to MHC-II, the binding for 27 types of HLA-II alleles accounting for 90% of all HLA-II alleles was predicted (http://tools.iedb.org/mhcii/result/).

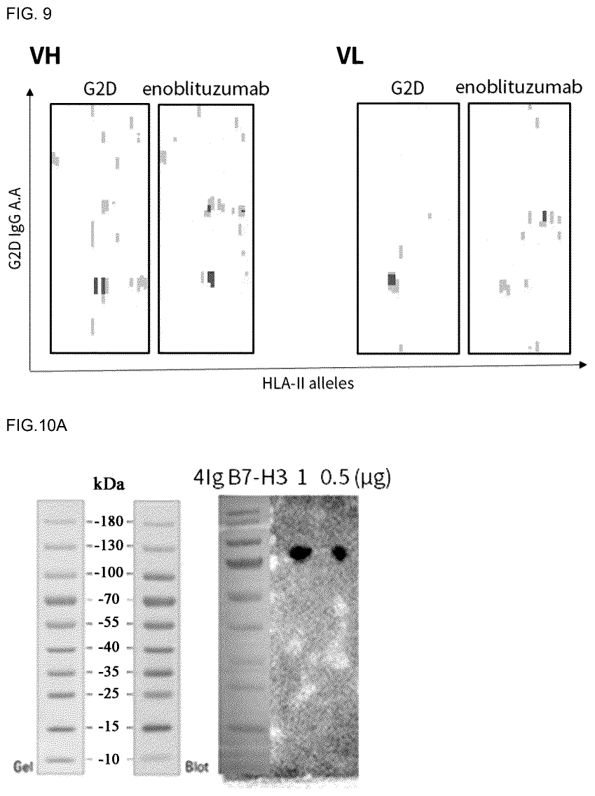

In FIG. 9, the HLA-II alleles are shown in the abscissa, and the 15 amino acids of G2D IgG with 14-amino acid overlaps are shown in the ordinate. Therefore, the possibility of binding of the 15 amino acids of G2D IgG for the HLA-II alleles can be evaluated. The result was displayed as red for <1 (possibility of binding: high), orange for 1 or greater and less than 5 (possibility of binding: medium), and yellow for 5 or greater and less than 10 (possibility of binding: low). It was confirmed that G2D IgG has comparable or lower possibility of immunogenicity as compared to the control antibody enoblituzumab (FIG. 9). This result suggests that G2D IgG has low possibility of inducing immune responses in the body.

### Example 7: Identification of Epitopes of G2D IgG

An epitope, also known as an antigen determinant, refers to a specific sequence of an antigen, which can be recognized by an antibody for binding. Since the therapeutic effect of an antibody and the degree of activating immune cells of an immuno-anticancer agent may vary depending on epitopes, the identification of the epitopes of an antibody is of great importance in drug development.

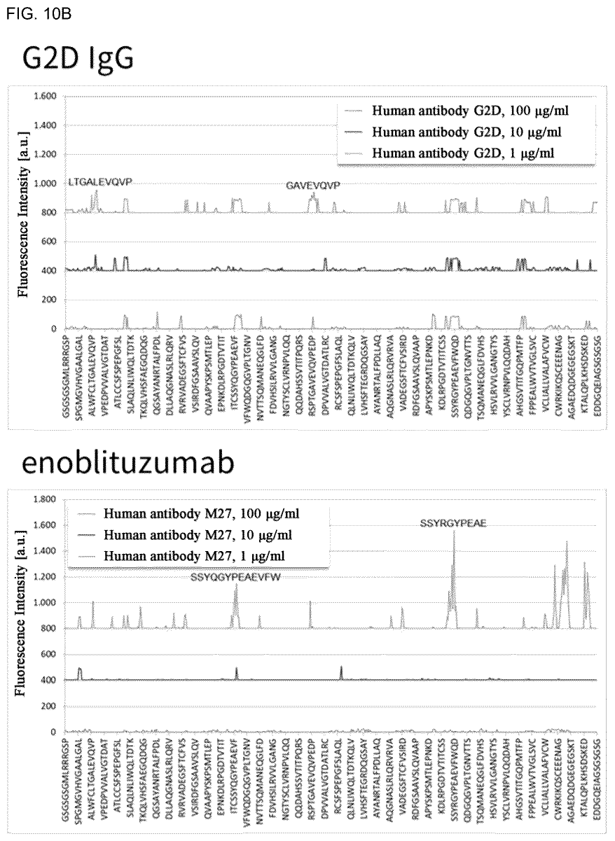

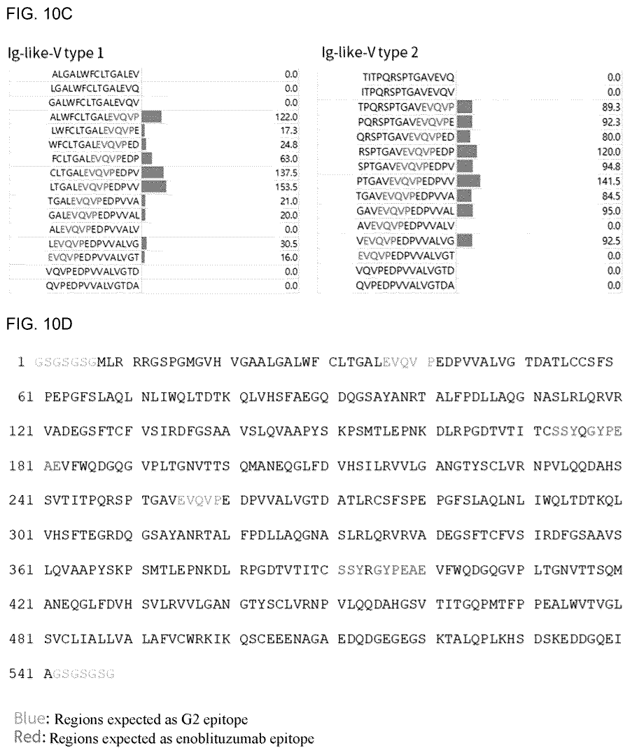

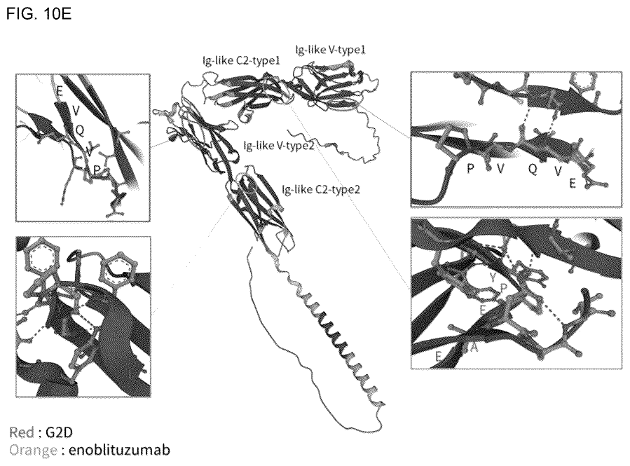

It was confirmed that G2D IgG has linear epitopes since G2D was verified to recognize 4lg B7-H3 through western blot (FIG. 10a). Therefore, linear epitope mapping was conducted by Pepperprint GmbH (Germany). After preparing a peptide microarray from the 15 4lg B7-H3 amino acids with 14-amino acid overlaps, the antibodies binding to peptides were identified using secondary antibodies (FIG. 10b). As a result, it was confirmed that G2D IgG binds to a peptide having a sequence of EVQVP (FIG. 10c). EVQVP is located in the Ig-like V-type1 and Ig-like V-type2 domains of 4lg B7-H3 (FIGS. 10d and 10e). When the epitopes of the control antibody enoblituzumab were identified by the same method, it was confirmed that the epitopes include the sequence of GYPEAE (FIG. 10b), and GYPEAE is located in the Ig-like C2-type1 and Ig-like C2-type2 domains of 4lg B7-H3 (FIGS. 10d and 10e). As a result, it was confirmed that G2D IgG and the control antibody enoblituzumab have different epitopes.

The sequence of B7-H3, its domain sequence, and the sequences of the epitopes to which the G2D antibody binds are as follows (Table 8).

### B7-H3 Sequence

Human 4Ig B7-H3
(SEQ ID NO: 33)
MLRRRGSPGMGVHVGAALGALWFCLTGALEVQVPEDPVVALVGTDATL
CCSFSPEPGFSLAQLNLIWQLTDTKQLVHSFAEGQDQGSAYANRTALF
PDLLAQGNASLRLQRVRVADEGSFTCFVSIRDFGSAAVSLQVAAPYSK
PSMTLEPNKDLRPGDTVTITCSSYQGYPEAEVFWQDGQGVPLTGNVTT
SQMANEQGLFDVHSILRVVLGANGTYSCLVRNPVLQQDAHSSVTITPQ
RSPTGAVEVQVPEDPVVALVGTDATLRCSFSPEPGFSLAQLNLIWQLT
DTKQLVHSFTEGRDQGSAYANRTALFPDLLAQGNASLRLQRVRVADEG
SFTCFVSIRDFGSAAVSLQVAAPYSKPSMTLEPNKDLRPGDTVTITCS
SYRGYPEAEVFWQDGQGVPLTGNVTTSQMANEQGLFDVHSVLRVVLGA
NGTYSCLVRNPVLQQDAHGSVTITGQPMTFPPEALWVTVGLSVCLIAL
LVALAFCWRKIKQSCEEENAGAEDQDGEGEGSKTALQPLKHSDSKEDD
GQEIA
Human 2Ig B7-H3
(SEQ ID NO: 34)
MLRRRGSPGMGVHVGAALGALWFCLTGALEVQVPEDPVVALVGTDATL
CCSFSPEPGFSLAQLNLIWQLTDTKQLVHSFAEGQDQGSAYANRTALF
PDLLAQGNASLRLQRVRVADEGSFTCFVSIRDFGSAAVSLQVAAPYSK
PSMTLEPNKDLRPGDTVTITCSSYRGYPEAEVFWQDGQGVPLTGNVTT
SQMANEQGLFDVHSVLRVVLGANGTYSCLVRNPVLQQDAHGSVTITGQ
PMTFPPEALWVTVGLSVCLIALLVALAFVCWRKIKQSCEEENAGAEDQ
DGEGEGSKTALQPLKHSDSKEDDGQEIA
Mouse B7-H3
(SEQ ID NO: 35)
MLRGWGGPSVGVCVRTALGVLCLCLTGAVEVQVSEDPVVALVDTDATL
RCSFSPEPGFSLAQLNLIWQLTDTKQLVHSFTEGRDQGSAYSNRTALF
PDLLVQGNASLRLQRVRVTDEGSYTCFVSIQDFDSAAVSQVAAPYSKP
SMTLEPNKDLRPGNMVTITCSSYQGYPEAEVFWKDGQGVPLTGNVTTS
QMANERGLFDVHSVLRVVLGANGTYSCLVRNPVLQQDAHGSVTITGQP
LTFPPEALWVTVGLSVCLVVLLVALAFVCWRKIKQSCEEENAGAEDQD
GDGEGSKTALRPLKPSENKEDDGQEIA

TABLE 8
Sequence of B7-H3 domains and location of epitopes

| | Domain | Sequence | SEQ ID NO |
|---|---|---|---|
| Human | Ig-like-V- | LEVQVPEDPV VALVGTDATL CCSFSPEPGF | 36 |
| 4Ig B7-H3 | type 1 | SLAQLNLIWQ LTDTKQLVHS FAEGQDQGSA | |
| | | YANRTALFPD LLAQGNASLR LQRVRVADEG | |
| | | SFTCFVSIRD FGSAAVSLQV A | |
| | Ig-like- | PSMTLEPNKD LRPGDTVTIT CSSYQGYPEA | 37 |
| | C2-type 1 | EVFWQDGQGV PLTGNVTTSQ MANEQGLFDV | |
| | | HSILRVVLGA NGTYSCLVRN PVLQQDAHSS | |
| | | VTIT | |
| | Ig-like-V- | PTGAVEVQVP EDPVVALVGT DATLRCSFSP | 38 |
| | type 2 | EPGFSLAQLN LIWQLTDTKQ LVHSFTEGRD | |
| | | QGSAYANRTA LFPDLLAQGN ASLRLQRVRV | |
| | | ADEGSFTCFV SIRDFGSAAV SLQVA | |
| | Ig-like- | PSMTLEPNKD LRPGDTVTIT CSSYRGYPEA | 39 |
| | C2-type 2 | EVFWQDGQGV PLTGNVTTSQ MANEQGLFDV | |
| | | HSVLRVVLGA NGTYSCLVRN PVLQQDAHGS | |
| | | VTIT | |
| Human | Ig-like-V- | LEVQVPEDPV VALVGTDATL CCSFSPEPGF | 36 |
| 2Ig B7-H3 | type 1 | SLAQLNLIWQ LTDTKQLVHS FAEGQDQGSA | |
| | | YANRTALFPD LLAQGNASLR LQRVRVADEG | |
| | | SFTCFVSIRD FGSAAVSLQV A | |
| | Ig-like- | PSMTLEPNKD LRPGDTVTIT CSSYRGYPEA | 39 |
| | C2-type 1 | EVFWQDGQGV PLTGNVTTSQ MANEQGLFDV | |
| | | HSVLRVVLGA NGTYSCLVRN PVLQQDAHGS | |
| | | VTIT | |
| | Ig-like V- | VEVQVSEDPV VALVDTDATL RCSFSPEPGF | 40 |
| | type | SLAQLNLIWQ LTDTKQLVHS FTEGRDQGSA | |
| | | YSNRTALFPD LLVQGNASLR LQRVRVTDEG | |
| | | SYTCFVSIQD FDSAAVSLQV A | |
| | Ig-like | PSMTLEPNKD LRPGNMVTIT CSSYQGYPEA | 41 |
| | C2-type | EVFWKDGQGV PLTGNVTTSQ MANERGLFDV | |
| | | HSVLRVVLGA NGTYSCLVRN PVLQQDAHGS | |
| | | VTIT | |

In the sequences, the underlined bold type indicates the epitopes of B7-H3 to which the G2D antibody binds.

### Example 8: Confirmation of T Cell-Mediated Cancer Cell-Killing Effect of G2D IgG

T cell-mediated cancer cell-killing effect was investigated using A549 cells which were confirmed to bind with G2D IgG (FIG. 7c). Enoblituzumab with wild-type Fc was used to compare the effect by B7-H3 binding. After adding 2×10⁵ PBMCs (STEMCELL Technologies, Canada), which were activated with an anti-CD3/CD28 antibody (eBioscience, USA), to 1×10⁴ GFP-inserted A549 cells, the cells were treated with 200 μg/mL G2D IgG and co-cultured for 48 hours. As a control group, PBMCs which were not activated were used under the same condition. After 48 hours of the co-culturing, the viability of the A549 cells was investigated by measuring the fluorescence of GFP with a SPARK instrument (TECAN, Switzerland). As a result, G2D IgG showed T cell-mediated cancer cell-killing effect of about 30% as compared to the negative control group, and the effect was comparable to or higher than that of the control antibody enoblituzumab with wild-type Fc (Table 9). This means that G2D IgG can enhance T cell-mediated killing effect by binding to B7-H3 on the cancer cell surface, and that G2D IgG can be used as an immuno-anticancer agent.

TABLE 9
T cell-mediated A549 cell-killing effect

| | $A 549 ability ( \% ) = \frac{A 549 viabilityof ( A 549 + PBMC + anti - CD 3 / CD 28 )}{A 549 viabilityof ( A 549 + PBMC )} \times 100$ |
|---|---|
| Negative | 100 |
| control | |
| group | |
| Isotype | 99.5 |
| control | |
| G2D | 69.3 |
| Eno- | 77.3 |
| blituzumab | |

### Example 9: Evaluation of Anticancer Efficacy of G2D IgG in GL261-Transplanted Syngeneic Mouse Model

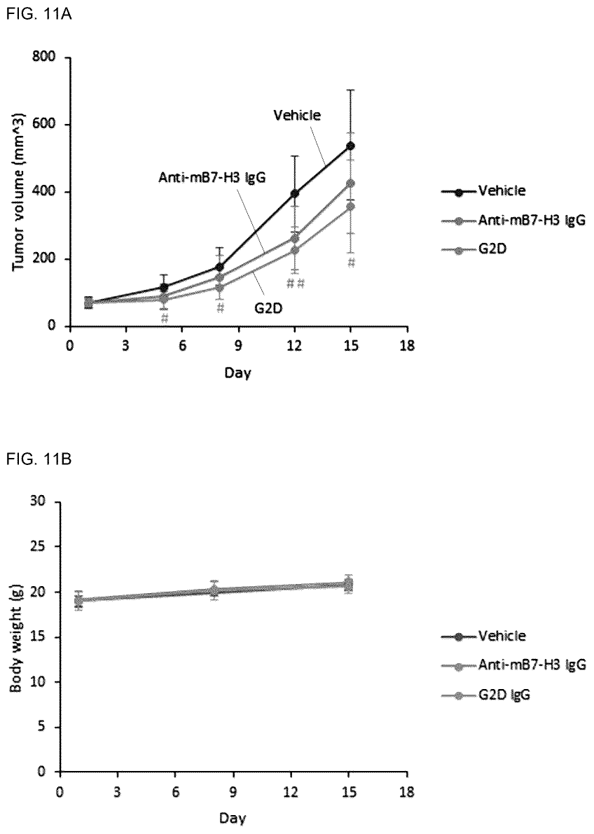

Since the potential of G2D IgG as an immuno-anticancer agent was confirmed in Example 8, it was verified using an animal model. Since a syngeneic mouse model induces cancer with the mouse immune system conserved, it is useful for verification of the anticancer effect by immune cells without transplantation of immune cells if the antibody to be evaluated can recognize the antigens of mouse cancer cells. GL261 mouse glioma cells, to which G2D IgG was confirmed to bind (FIG. 8), were transplanted into the hypoderm of the right flank of immunocompetent female C57BL/6 mice (Orient Bio, Korea) at 4×10⁶ cells/mouse. The administration of G2D IgG was started when the tumor size reached about 70 mm³. As a control drug, InVivoMAb anti-mouse CD276 (anti-mB7-H3 IgG) (BioXcell, USA), which is an antibody binding to mouse B7-H3, was used. The antibody was intravenously injected at a concentration of 10 mg/kg for 2 weeks, twice a week. Tumor volume measurement was conducted for 2 weeks, twice a week, after the drug administration was started. The tumor volume (mm³) was calculated from the equation, [tumor volume (mm³)=(major axis×minor axis²)/2]. Body weight was measured using an electronic scale once a week after the starting of the drug administration and on the day of autopsy. Statistical analysis was conducted by Student's t-test, and p<0.05 was considered statistically significant (#). As a result, G2D IgG showed larger decrease in tumor volume without body weight loss, as compared to the control drug, anti-mB7-H3 IgG (FIG. 11). This result suggests that G2D IgG exhibits anticancer efficacy by effectively inhibiting B7-H3.

Although the specific exemplary embodiments of the present disclosure have described, those having ordinary knowledge in the art can make various modifications and changes through addition, change, deletion, etc. of elements without departing from the scope of the present disclosure.

1. An anti-B7-H3 antibody or an antigen-binding fragment thereof, which specifically binds to an epitope located within the amino acid sequence of a domain of B7-H3, selected from a group consisting of an Ig-like-V-type 1 sequence of SEQ ID NO: 36, an Ig-like-V-type 2 sequence of SEQ ID NO: 38 and an Ig-like-V-type sequence of SEQ ID NO: 40.

2. The anti-B7-H3 antibody or the antigen-binding fragment thereof according to claim 1, wherein the anti-B7-H3 antibody or the antigen-binding fragment thereof specifically binds to one or more amino acids of amino acid residues 2 to 6 of SEQ ID NO: 36 (i.e., EVQVP), amino acid residues 6 to 10 of SEQ ID NO: 38 (i.e., EVQVP) and amino acid residue 2 to 6 of SEQ ID NO: 40 (i.e., EVQVS).

3. The anti-B7-H3 antibody or the antigen-binding fragment thereof according to claim 1, wherein the anti-B7-H3 antibody or the antigen-binding fragment thereof comprises light chain CDR1, CDR2 and CDR3 and heavy chain CDR1, CDR2 and CDR3, wherein the light chain CDR1, CDR2 and CDR3 are represented by SEQ ID NOS: 3, 4 and 5, respectively, and the heavy chain CDR1, CDR2 and CDR3 are represented by SEQ ID NOS: 6, 7 and 8, respectively.

4. An anti-B7-H3 antibody or an antigen-binding fragment thereof binding specifically to B7-H3, wherein
the anti-B7-H3 antibody or the antigen-binding fragment thereof comprises light chain CDR1, CDR2 and CDR3 and heavy chain CDR1, CDR2 and CDR3, wherein the light chain CDR1, CDR2 and CDR3 are represented by SEQ ID NOS: 3, 4 and 5, respectively, and the heavy chain CDR1, CDR2 and CDR3 are represented by SEQ ID NOS: 6, 7 and 8, respectively.

5. The anti-B7-H3 antibody or the antigen-binding fragment thereof according to claim 3 or 4, wherein
the anti-B7-H3 antibody or the antigen-binding fragment thereof comprises a light chain variable region and a heavy chain variable region, and
(i) the light chain variable region comprises an amino acid sequence which is identical to a sequence represented by SEQ ID NO: 9 by at least 80%, at least 85%, at least 90%, at least 95%, at least 96%, at least 97%, at least 98%, at least 99%, or 100%; and
(ii) the heavy chain variable region comprises an amino acid sequence which is identical to a sequence represented by SEQ ID NO: 10 by at least 80%, at least 99%, or 100%.

6. The anti-B7-H3 antibody or the antigen-binding fragment thereof according to claim 5, wherein
the anti-B7-H3 antibody or the antigen-binding fragment thereof comprises a light chain variable region and a heavy chain variable region, and
the light chain variable region is represented by SEQ ID NO: 9, and the heavy chain variable region is represented by SEQ ID NO: 10.

7. The anti-B7-H3 antibody or the antigen-binding fragment thereof according to claim 3 or 4, wherein
the anti-B7-H3 antibody or the antigen-binding fragment thereof comprises a light chain variable region and a heavy chain variable region, and
(i) the light chain variable region comprises an amino acid sequence which is identical to a sequence represented by SEQ ID NO: 21 by at least 80%, at least 85%, at least 90%, at least 95%, at least 96%, at least 97%, at least 98%, at least 99%, or 100%; and
(ii) the heavy chain variable region comprises an amino acid sequence which is identical to a sequence represented by SEQ ID NO: 22 by at least 80%, at least 99%, or 100%.

8. The anti-B7-H3 antibody or the antigen-binding fragment thereof according to claim 7, wherein
the anti-B7-H3 antibody or the antigen-binding fragment thereof comprises a light chain variable region and a heavy chain variable region, and
the light chain variable region is represented by SEQ ID NO: 21, and the heavy chain variable region is represented by SEQ ID NO: 22.

9. A nucleic acid encoding the anti-B7-H3 antibody or the antigen-binding fragment thereof according to claim 1 or 4.

10. A vector comprising the nucleic acid according to claim 9.

11. A cell comprising the vector according to claim 10.

12. A cell transformed with the vector according to claim 10.

13. A composition comprising an ingredient selected from a group consisting of the anti-B7-H3 antibody or the antigen-binding fragment thereof according to claim 1 or 4, nucleic acid encoding the antibody or the antigen-binding fragment thereof, a vector comprising the nucleic acid, and a cell comprising the vector or transformed with the vector.

14. A pharmaceutical composition for preventing or treating cancer, comprising an ingredient selected from a group consisting of the anti-B7-H3 antibody or the antigen-binding fragment thereof according to claim 1 or 4, nucleic acid encoding the antibody or the antigen-binding fragment thereof, a vector comprising the nucleic acid, and a cell comprising the vector or transformed with the vector, and a pharmaceutically acceptable carrier.

15. The pharmaceutical composition for preventing or treating cancer according to claim 14, wherein the cancer expresses B7-H3.

16. The pharmaceutical composition for preventing or treating cancer according to claim 14, wherein the cancer is selected from a group consisting of brain tumor, childhood cancer, medulloblastoma, leptomeningeal metastatic cancer, breast cancer, lung cancer, pancreatic cancer, bowel cancer, liver cancer, prostate cancer, ovarian cancer, stomach cancer, esophageal cancer, lymphoma, melanoma, kidney cancer, fibrosarcoma, colon cancer, colorectal cancer, endometrial cancer, thyroid cancer, parathyroid cancer, cervical cancer, bladder cancer, head and neck cancer, bone cancer, skin cancer, uterine cancer, testicular cancer, bile duct cancer, bronchial cancer, nasopharyngeal cancer, laryngeal cancer and ureteral cancer.

17. The pharmaceutical composition for preventing or treating cancer according to claim 14, wherein the pharmaceutical composition is used in combination with an additional anticancer agent comprising an immunotherapeutic agent, a chemotherapeutic agent, a targeted therapeutic agent, or a radiotherapeutic agent.

18. A method for treating cancer, comprising a step of administering an ingredient selected from a group consisting of the anti-B7-H3 antibody or the antigen-binding fragment thereof according to claim 1 or 4, a nucleic acid encoding the antibody or the antigen-binding fragment thereof, a vector comprising the nucleic acid, and a cell comprising the vector or transformed with the vector to a subject in need thereof.

19. A medical use of an ingredient selected from a group consisting of the anti-B7-H3 antibody or the antigen-binding fragment thereof according to claim 1 or 4, a nucleic acid encoding the antibody or the antigen-binding fragment thereof, a vector comprising the nucleic acid, and a cell comprising the vector or transformed with the 5 vector.

20. A composition for diagnosing cancer, comprising an ingredient selected from a group consisting of the anti-B7-H3 antibody or the antigen-binding fragment thereof according to claim 1 or 4, a nucleic acid encoding the antibody or the antigen-binding fragment thereof, a vector comprising the nucleic acid, and a cell comprising the vector or transformed with the vector.


SEQUENCE LISTING (41 sequences)

SEQ ID NO: 1 - DNA, 18 bp, synthetic construct
  misc_feature 1..18: note = Primer
  source 1..18: mol_type = other DNA
acactttatgcttccggc

SEQ ID NO: 2 - DNA, 20 bp, synthetic construct
  misc_feature 1..20: note = Primer
  source 1..20: mol_type = other DNA
caaaatcaccggaaccagag

SEQ ID NO: 3 - protein, 8 aa, synthetic construct
  REGION 1..8: note = LCDR1
SGGSIGYG

SEQ ID NO: 4 - protein, 7 aa, synthetic construct
  REGION 1..7: note = LCDR2
YNDRRPS

SEQ ID NO: 5 - protein, 12 aa, synthetic construct
  REGION 1..12: note = LCDR3
GSADSSSTYTGI

SEQ ID NO: 6 - protein, 5 aa, synthetic construct
  REGION 1..5: note = HCDR1
SYPMV

SEQ ID NO: 7 - protein, 17 aa, synthetic construct
  REGION 1..17: note = HCDR2
SINSGGSWTGYGAAVKG

SEQ ID NO: 8 - protein, 9 aa, synthetic construct
  REGION 1..9: note = HCDR3
AYGAATIDA

SEQ ID NO: 9 - protein, 105 aa, synthetic construct
  REGION 1..105: note = VL_2D
ALTQPSSVSANLGGTVKITCSGGSIGYGWYQQKAPGSAPVTVIYYNDRRPSDIPSRFSGSKSGSANTLTITGVQADDEAIYYCGSADSSSTYTGIFGAGTTLTVL

SEQ ID NO: 10 - protein, 118 aa, synthetic construct
  REGION 1..118: note = VH_2D
AVTLDESGGGLQTPGGGLSLVCKASGFDFSSYPMVWVRQAPGKGLEYVASINSGGSWTGYGAAVKGRATISRDNGQSTVRLQLNNLRAEDTATYYCARAYGAATIDAWGHGTEVIVSS

SEQ ID NO: 11 - DNA, 59 bp, synthetic construct
  misc_feature 1..59: note = Primer
  source 1..59: mol_type = other DNA
ggtctttgtatacatgttgctgtggttgtctggtgttgaaggagccctgactcagccgt

SEQ ID NO: 12 - DNA, 27 bp, synthetic construct
  misc_feature 1..27: note = Primer
  source 1..27: mol_type = other DNA
ggccacggtccgtaggacggtcagggt

SEQ ID NO: 13 - DNA, 20 bp, synthetic construct
  misc_feature 1..20: note = Primer
  source 1..20: mol_type = other DNA
cggaccgtggccgccccctc

SEQ ID NO: 14 - DNA, 26 bp, synthetic construct
  misc_feature 1..26: note = Primer
  source 1..26: mol_type = other DNA
tagttctagaactagcactcgccccg

SEQ ID NO: 15 - DNA, 100 bp, synthetic construct
  misc_feature 1..100: note = Primer
  source 1..100: mol_type = other DNA
gggaattctagaggatcgaaccctttgcaagcttcggcacgagcagaccagcatgggcatcaagatggagacacattctcaggtctttgtatacatgttg

SEQ ID NO: 16 - DNA, 46 bp, synthetic construct
  misc_feature 1..46: note = Primer
  source 1..46: mol_type = other DNA
cttcctgtcagtaactacaggtgtccactccgccgtgacgttggac

SEQ ID NO: 17 - DNA, 29 bp, synthetic construct
  misc_feature 1..29: note = Primer
  source 1..29: mol_type = other DNA
cccttggtggaggcggaggagacgatgac

SEQ ID NO: 18 - DNA, 17 bp, synthetic construct
  misc_feature 1..17: note = Primer
  source 1..17: mol_type = other DNA
gcctccaccaagggccc

SEQ ID NO: 19 - DNA, 27 bp, synthetic construct
  misc_feature 1..27: note = Primer
  source 1..27: mol_type = other DNA
gtgagcggccgctcacttgccggggga

SEQ ID NO: 20 - DNA, 63 bp, synthetic construct
  misc_feature 1..63: note = Primer
  source 1..63: mol_type = other DNA
cagaattcactctaaccatggaatggagctgggtctttctcttcttcctgtcagtaactacag

SEQ ID NO: 21 - protein, 107 aa, synthetic construct
  REGION 1..107: note = VL-G2D
SYELTQPPSVSVSPGQTARITCSGGSIGYGWYQQKAPGQAPVTVIYYNDRRPSGIPERFSGSKSGTTNTLTISGVQAEDEADYYCGSADSSSTYTGIFGGGTKLTVL

SEQ ID NO: 22 - protein, 118 aa, synthetic construct
  REGION 1..118: note = VH-G2D
QVQLVESGGGLVQPGGSLRLSCSASGFDFSSYPMVWVRQAPGKGLEYVSSINSGGSWTGYGAAVKGRATISRDNSKNTLYLQMNSLRAEDTATYYCARAYGAATIDAWGQGTTVTVSS

SEQ ID NO: 23 - DNA, 66 bp, synthetic construct
  misc_feature 1..66: note = Primer
  source 1..66: mol_type = other DNA
tctcaggtctttgtatacatgttgctgtggttgtctggtgttgaaggatcctacgagttgacgcag

SEQ ID NO: 24 - DNA, 37 bp, synthetic construct
  misc_feature 1..37: note = Primer
  source 1..37: mol_type = other DNA
gggcggccacggtccggagaactgtcagtttcgttcc

SEQ ID NO: 25 - DNA, 20 bp, synthetic construct
  misc_feature 1..20: note = Primer
  source 1..20: mol_type = other DNA
cggaccgtggccgccccctc

SEQ ID NO: 26 - DNA, 26 bp, synthetic construct
  misc_feature 1..26: note = Primer
  source 1..26: mol_type = other DNA
tagttctagaactagcactcgccccg

SEQ ID NO: 27 - DNA, 100 bp, synthetic construct; identical to SEQ ID NO: 15
  misc_feature 1..100: note = Primer
  source 1..100: mol_type = other DNA

SEQ ID NO: 28 - DNA, 51 bp, synthetic construct
  misc_feature 1..51: note = Primer
  source 1..51: mol_type = other DNA
cttcctgtcagtaactacaggtgtccactcccaggtccagctggttgaaag

SEQ ID NO: 29 - DNA, 36 bp, synthetic construct
  misc_feature 1..36: note = Primer
  source 1..36: mol_type = other DNA
ggcccttggtggaggcgctagacactgtcacggttg

SEQ ID NO: 30 - DNA, 17 bp, synthetic construct
  misc_feature 1..17: note = Primer
  source 1..17: mol_type = other DNA
gcctccaccaagggccc

SEQ ID NO: 31 - DNA, 27 bp, synthetic construct
  misc_feature 1..27: note = Primer
  source 1..27: mol_type = other DNA
gtgagcggccgctcacttgccggggga

SEQ ID NO: 32 - DNA, 63 bp, synthetic construct; identical to SEQ ID NO: 20
  misc_feature 1..63: note = Primer
  source 1..63: mol_type = other DNA

SEQ ID NO: 33 - protein, 533 aa, synthetic construct
  REGION 1..533: note = Human 4Ig B7-H3
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFCWRKIKQSCEEENAGAEDQDGEGEGSKTALQPLKHSDSKEDDGQEIA

SEQ ID NO: 34 - protein, 316 aa, synthetic construct
  REGION 1..316: note = Human 2Ig B7-H3
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 35 - protein, 315 aa, synthetic construct
  REGION 1..315: note = Mouse B7-H3
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 36 - protein, 111 aa, synthetic construct
  REGION 1..111: note = Ig-like-V-type 1_Human B7-H3
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVA

SEQ ID NO: 37 - protein, 94 aa, synthetic construct
  REGION 1..94: note = Ig-like-C2-type 1_Human 4Ig B7-H3
PSMTLEPNKDLRPGDTVTITCSSYQGYPEAEVFWQDGQGVPLTGNVTTSQMANEQGLFDVHSILRVVLGANGTYSCLVRNPVLQQDAHSSVTIT

SEQ ID NO: 38 - protein, 115 aa, synthetic construct
  REGION 1..115: note = Ig-like-V-type 2_Human 4Ig B7-H3
PTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVA

SEQ ID NO: 39 - protein, 94 aa, synthetic construct
  REGION 1..94: note = Ig-like-C2-type 2_Human 4Ig B7-H3 or Ig-like-C2-type 1_Human 2Ig B7-H3
PSMTLEPNKDLRPGDTVTITCSSYRGYPEAEVFWQDGQGVPLTGNVTTSQMANEQGLFDVHSVLRVVLGANGTYSCLVRNPVLQQDAHGSVTIT

SEQ ID NO: 40 - protein, 111 aa, synthetic construct
  REGION 1..111: note = Ig-like V-type_Mouse B7-H3
VEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVA

SEQ ID NO: 41 - protein, 94 aa, synthetic construct
  REGION 1..94: note = Ig-like C2-type_Mouse B7-H3
PSMTLEPNKDLRPGNMVTITCSSYQGYPEAEVFWKDGQGVPLTGNVTTSQMANERGLFDVHSVLRVVLGANGTYSCLVRNPVLQQDAHGSVTIT